(19) United States
(12) Patent Application Publication
(10) Pub. No.: US 2026/0271966 A1
(43) Pub. Date: Sep. 17, 2026

(54) PROBIOTICS TO REDUCE GREENHOUSE GAS EMISSIONS IN CATTLE

(71) Applicant: BiomEdit, Inc., Fishers, IN (US)

(72) Inventors: Arvind KUMAR, Fishers, IN (US); Diwi SUSANTI, Fishers, IN (US)

(21) Appl. No.: 19/167,017

(22) PCT Filed: Mar. 21, 2024
(86) PCT No.: PCT/US2024/020945

§ 371 Date: Sep. 19, 2025

Related U.S. Application Data
(60) Provisional application No. 63/453,894, filed on Mar. 22, 2023.

(51) Int. Cl. A23K 10/18; A23K 10/12; A23K 50/10

(52) U.S. Cl. CPC A23K 10/18; A23K 10/12; A23K 50/10

(57) ABSTRACT

The present invention relates to probiotic or microbiome modulator compositions and methods for reducing greenhouse gas emissions, such as methane emissions, that use such compositions. The probiotic or microbiome modulator compositions include one or more isolated bacterial strains which alter the microbiome and/or alter or kill methanogenic bacteria.

### SEQUENCE LISTING XML

The instant application contains a Sequence Listing encoded in XML format which was filed electronically by EFS-web and is hereby incorporated by reference in its entirety. Said XML format Sequence Listing, created on Mar. 21, 2023, is named “2950-24_P1_ST26.XML” and is 18,761,302 bytes in size.

### FIELD

The present disclosure relates to probiotic or microbiome modulator compositions and methods for reducing greenhouse gas emissions, such as methane emissions. The probiotic compositions or microbiome modulator include one or more isolated bacterial strains which reduce greenhouse gas and methane emissions, alter, disrupt or kill methanogenic Archaea, and modulate microbiota composition structures.

### BACKGROUND

Methane is produced as a by-product of ruminal microbial fermentation process. In particular, H₂ and CO₂ are byproducts of the fermentation process and utilized by methanogenic Archaea (methanogens) to form methane. 40% of methane is from natural sources, such as animals. The remaining 60% is from human sources including fossil fuels, landfills and biomass burning. In the US alone, livestock production emits close to 200 million metric tons of CO₂-equivalent of methane. Much of the methane from livestock is generated in the rumen of ruminant animals by methanogens, with only a small amount emitted from hind-gut fermentation in ruminant and non-ruminant animals. Methane production and emission results in a 6-12% energy loss for the livestock animals.

Methanogens, unique microbes from the Archaeal domain, are responsible for methane production via the methanogenesis pathway which reduces carbon dioxide, methanol, or methylamines to methane, using electrons from hydrogens. They are common in wetlands, where they are responsible for marsh gas. Examples of methane-producing genera are Methanobrevibacter, Methanobacterium, Methanosarcina, Methanococcus, and Methanospirillum. Methanogenic organisms are widespread in anaerobic nature and are found in mud, sewage, and sludge and in the gastrointestinal tract of animals. Methanogens typically thrive in environments in which all other electron acceptors (such as nitrate, sulfate, and trivalent iron) are limited. Methanogenic organisms, particularly methanogenic Archaea are found in the digestive tracts of animals such as ruminants and non-ruminant animals including humans, where they are responsible for the methane content of belching in ruminants and flatulence in humans. Ruminants are large hoofed herbivorous grazing or browsing mammals that are able to acquire nutrients from plant-based food by fermenting it in a specialized stomach prior to digestion, principally through microbial actions. In marine sediments, the biological production of methane is generally confined to where sulfates are depleted, below the top layers and in the marine sediment or low layers which are anaerobic. Moreover, methanogenic Archaea populations play an indispensable role in anaerobic wastewater treatments and global carbon cycles. Others are extremophiles, found in environments such as hot springs and submarine hydrothermal vents as well as in the “solid” rock of Earth's crust, kilometers below the surface.

The global population will increase by 33% in 2050 to 9.6 billion. The projected meat and milk protein demand will rise to 73 and 58%, respectively, compared to those in 2010 (FAO). See Tackling climate change through livestock, FAO, 2013. An increase in livestock productions is expected to make a significant contribution to global climate change (GHG emissions, N₂O, CO₂, CH₄), as livestock GHG have accounted for 18% of global emissions. Among GHG, methane has a shorter shelf life and is 28-times more potent than CO₂. Enteric methane emissions account for 44.3% of GHG emissions from livestock production. A total of 100 Mton CH₄ is emitted per year. Moreover, methane emission is considered loss of energy for animals. Methane emissions can result in a 6-12% energy loss for animals.

There are net zero initiatives in the cattle industry and market opportunities for the reduction of enteric CH₄ emissions. Net zero pledges from supply chains puts pressure on cattle industries to meet sustainability/greenhouse gas (GHG) reduction goal(s). In addition, a government incentive program providing for a carbon credit can provide an incentive to reduce methane emissions (for example President Biden's administration 2030 aggressive goal for reduction of GHG in the US).

Direct fed microbials (DFMs), often also called probiotics or microbiome modulators, are microorganisms which colonize the gastrointestinal tract of an animal and provide some beneficial effect to that animal. The microbials can be bacterial species, for example those from the genera Bacillus, Lactobacillus, Lactococcus, and Enterococcus. The microorganisms can also be yeast or even molds. The microorganisms can be provided to an animal orally, such as in feed, or by other administration methods including, in the case of birds, provided to a fertilized egg, i.e. in ovo.

A beneficial activity provided by a DFM can be through the synthesis and secretion of vitamins or other nutritional molecules needed for a healthy metabolism of the host animal or by production of agents or molecules which inhibit or effect other organisms, microbials or bacteria. DFMs can also alter the microbiome, either in an animal or where microorganisms or bacteria are present. A DFM can protect the host animal from disease, disorders, or clinical symptoms caused by microorganisms or other agents. For example, the DFM may naturally produce factors having inhibitory or killing activity against certain species of pathogens, such as deleterious or disease-causing bacteria. A DFM can also alter the microbiome in an animal resulting in beneficial effects, particularly as the microbiome and its components is connected to various diseases, conditions or activities in an animal or host. Probiotics and DFMs provide an attractive alternative or addition for disease prevention and/or to the use and application of antibiotics in animals. Antibiotics can promote resistant or less sensitive bacteria and can ultimately end up in feed products or foods consumed by other animals or humans posing greater public health crisis. DFMs are characterized as being generally safe, even denoted Generally Regarded as Safe (GRAS), and most are not naturally resistant to antibiotics.

There is a need in the art for probiotic and microbiome modulator compositions and to provide beneficial molecules to the gastrointestinal tract of an animal and thus improve animal health. There is a need to reduce GHG emissions. There is a need in the art for probiotic and microbiome modulator compositions with capability to reduce GHG emissions resulting from microorganisms, including Archaca and bacteria, both in animals and in the environment. In particular, there is a need to reduce methane emissions from animals such as livestock and, particularly, ruminant animals, or from natural sources where methanogenic Archaea reside.

### SUMMARY OF THE INVENTION

The present disclosure provides compositions and methods for reducing greenhouse gas emissions, particularly methane emissions generated by methanogenic organisms such as methanogenic Archaea or from animals or other sources where methanogenic organisms such as methanogenic Archaea reside. Such compositions and methods include at least one bacterial strain and can include combinations of bacterial strains.

The present disclosure provides compositions which include at least one strain selected from Bacillus spp. and/or Lactobacillus spp. as a probiotic or microbiome modulator, or combinations of such strains as probiotics or microbiome modulators. The present disclosure provides compositions which include at least one strain selected from Bacillus spp. and/or Lactobacillus spp. as a anti-methanogen or methanogenesis modulator, or combinations of such strains as anti-methanogens or methanogenesis modulators. The disclosure provides methods for reducing greenhouse gas emissions, particularly methane emissions, using such compositions.

The present disclosure provides a composition including at least one probiotic or microbiome modulator selected from a Bacillus spp. and Lactobacillus spp. or combinations thereof, wherein the composition reduces methane gas emissions, such as deriving from or in the presence of methanogenic Archaea organisms, when an effective amount is combined with one or more methanogenic Archaea organism or is administered to an animal harboring one or more methanogenic Archaea organism as compared to the one or more methanogenic Archaea organism in the absence of the composition or compared to an animal not administered the composition. The present disclosure provides a composition including at least one probiotic or microbiome modulator selected from a Bacillus spp. and Lactobacillus spp. or combinations thereof, wherein the composition reduces methane gas emissions from a ruminant when an effective amount is administered to the ruminant as compared to a ruminant not administered the composition. The present disclosure provides a composition comprising one or more probiotic or microbiome modulator bacteria, wherein the bacteria is selected from Bacillus spp. bacteria and Lactobacillus spp. bacteria, wherein the composition inhibits or kills one or more bacteria which is methanogenic. The composition thereby serves to reduce methane gas emissions from an animal, particularly including a ruminant, when an effective amount is administered to the animal, or particularly ruminant, as compared to an animal or ruminant not administered the composition. In an embodiment, the bacteria selected from Bacillus spp. bacteria and Lactobacillus spp. bacteria in the composition alters the microbiome or otherwise modulates or alters the environment of the one or more methanogenic organism, particularly one or more methanogenic Archaea. Methane production is thereby reduced in the presence and after administration or addition of the bacteria selected from Bacillus spp. bacteria and Lactobacillus spp. bacteria.

In an embodiment the one or more methanogenic organism is an organism that produces methane as a byproduct of their metabolism. In an embodiment the one or more methanogenic organism is a methanogenic Archaea. In an embodiment, the methanogenic Archaeaon is of the Methanobacteriales, the Methanomicrobiales, or the Methanosarcinales order. In an embodiment, the methanogenic Archaeaon is one or more Methanobacterium, Methanobrevibacter, Methanococcus, Methanosarcina, Methanosphaera, Methanomassilicoccales Archaea. In an embodiment, the methanogenic Archaeaon is Methanobrevibacter ruminantium, Methanobrevibacter gottschalkii, Methanosphaera stadtmanae, Methanomicrobium mobile, Methanomassilicoccaceae spp. Methanobacterium bryantii or Methanosarcina mazei. In an embodiment, the fermentative organism is Fibrobacter, Ruminococcus, Streptococcus bovis or Ruminobacter.

The present disclosure provides a method for reducing methane gas emissions from an animal including administering an effective amount of a composition including at least one probiotic or microbiome modulator selected from a Bacillus spp. and Lactobacillus spp. or combinations thereof, to the animal. The present disclosure provides a method for reducing methane gas emissions from a ruminant including administering an effective amount of a composition including at least one probiotic or microbiome modulator selected from a Bacillus spp. and Lactobacillus spp. or combinations thereof, to the ruminant.

In embodiments, methods are provided for reducing methane emission or production, in instances by methanogenic organisms, in manure, in ponds, in an aerobic digester. In embodiments, methods are provided for reducing methane emission or production, in instances by methanogenic organisms, in manure by combining or adding to the manure one or more compositions or fermentation product of one or more compositions, including the probiotic or microbiome modulator compositions provided herein. In embodiments, methods are provided for reducing methane emission or production, in instances by methanogenic organisms, in manure by combining or adding to the manure one or more compositions or fermentation product of one or more compositions, including the anti-methanogen or methanogenesis modulator compositions provided herein.

Bacterial strains, particularly Bacillus strains and Lactobacillus strains, are provided with capability and activity to reduce methane gas production. In an embodiment, bacterial strains are provided with capability and activity to alter the microbiome and the microorganisms and to reduce methane production and/or methanogenesis. In an embodiment, bacterial strains are provided with capability and activity to alter the microbiome and the fermentation facilitated by microorganisms so as to reduce methane production and/or methanogenesis. In an embodiment, bacterial strains are provided with capability and activity to modulate or alter the colonization of methanogenic organisms including methanogenic Archaea. In an embodiment, bacterial strains are provided with capability and activity to reduce growth or inhibit or alter colonization of methanogenic organisms including methanogenic Archaea. In an embodiment, bacterial strains are provided with capability and activity to reduce methane gas production in an animal. In an embodiment, bacterial strains are provided with capability and activity to reduce methane gas production in a ruminant animal. In an embodiment, bacterial strains are provided with capability and activity to reduce methane gas production in a non-ruminant animal. In an embodiment, bacterial strains are provided with capability and activity to inhibit or otherwise alter methanogenic Archaea and to alter methanogenesis in an animal.

In embodiments, the animal is a ruminant animal. In some embodiments, the animal is a non ruminant animal.

In an embodiment, bacterial strains are provided with capability and activity to reduce methane gas production in a ruminant animal. In an embodiment, bacterial strains are provided with capability and activity to reduce methane gas production in a ruminant or non-ruminant animal. In an embodiment, bacterial strains are provided with capability to alter the microbiome and modulate, including inhibit, methanogenesis and methane production due to methanogenic Archaea in an animal. In an embodiment, bacterial strains are provided with capability to alter the microbiome and modulate, including inhibit, methanogenesis and methane production due to methanogenic Archaea in a ruminant animal. In an embodiment, bacterial strains are provided with capability and activity to reduce growth or inhibit colonization of methanogenic bacteria in a ruminant animal.

In an embodiment, bacterial strains are provided with capability and activity to reduce methane gas production from native methanogenic Archaea in an animal. In an embodiment, bacterial strains are provided with capability and activity to reduce methane gas production from native methanogenic Archaea in the rumen of a ruminant animal. In an embodiment, bacterial strains are provided with capability and activity to inhibit methanogenic Archaea and/or alter methanogenesis via the microbiome and methanogenic organisms in the rumen of a ruminant animal. In an embodiment, bacterial strains are provided with capability and activity to reduce growth or inhibit colonization of methanogenic bacteria in the rumen of a ruminant animal.

In an embodiment, a Bacillus bacterial strain is provided with capability and activity to reduce methane gas production from native methanogenic Archaea or methanogenic bacteria in an animal. In an embodiment, one or more Bacillus bacterial strains are provided with capability and activity to reduce methane gas production from native methanogenic Archaea or bacteria in an animal. In an embodiment, a combination of one or more Bacillus bacterial strains are provided with capability and activity to reduce methane gas production from native methanogenic Archaea or bacteria in an animal. In an embodiment, Bacillus bacterial strains are provided with capability and activity to reduce methane gas production from native methanogenic Archaea or bacteria in the rumen of a ruminant animal. In an embodiment, one or more Bacillus strain is provided with capability and activity to inhibit methanogenic archaea and/or bacteria in an animal. In an embodiment, a Bacillus strain is provided with capability and activity to reduce methanogenesis via methanogenic Archaea in an animal. In an embodiment, a Bacillus strain is provided with capability and activity to reduce growth or inhibit colonization of methanogenic Archaea and/or methanogenic bacteria in an animal. In an embodiment, Bacillus bacterial strains are provided with capability and activity to inhibit methanogenic Archaea and/or methanogenic bacteria or to alter the microbiome comprising methanogenic Archaea and/or methanogenic bacteria in the rumen of a ruminant animal. In an embodiment, Bacillus bacterial strains are provided with capability and activity to reduce growth or inhibit colonization of methanogenic Archaea and/or methanogenic bacteria in the rumen of a ruminant animal.

In an embodiment, a Lactobacillus bacterial strain is provided with capability and activity to reduce methane gas production from native methanogenic Archaea or bacteria in an animal. In an embodiment, one or more Lactobacillus bacterial strains are provided with capability and activity to reduce methane gas production from native methanogenic Archaea or bacteria in an animal. In an embodiment, one or more Lactobacillus bacterial strains and one or more Bacillus bacterial strains in combination are provided with capability and activity to reduce methane gas production from native methanogenic Archaea or bacteria in an animal. In an embodiment, Lactobacillus bacterial strains are provided with capability and activity to reduce methane gas production from native methanogenic Archaea or bacteria in the rumen of a ruminant animal. In an embodiment, one or more Lactobacillus strain is provided with capability and activity to inhibit methanogenic archaea and/or bacteria in an animal. In an embodiment, a Lactobacillus strain is provided with capability and activity to reduce methanogenesis via methanogenic Archaea and/or bacteria in an animal. In an embodiment, a Lactobacillus strain is provided with capability and activity to reduce growth or inhibit colonization of methanogenic Archaea and/or methanogenic bacteria in an animal. In an embodiment, Lactobacillus bacterial strains are provided with capability and activity to inhibit methanogenic Archaea and/or methanogenic bacteria or to alter the microbiome comprising methanogenic Archaea and/or methanogenic bacteria in the rumen of a ruminant animal. In an embodiment, Lactobacillus bacterial strains are provided with capability and activity to reduce growth or inhibit colonization of methanogenic Archaea and/or methanogenic bacteria in the rumen of a ruminant animal.

In some embodiments, one or more Bacillus or Lactobacillus strain is provided with capability and activity to reduce methane gas production and/or with capability and activity to inhibit and/or with capability to alter the microbiome with includes methanogenic Archaea. In some embodiments, combinations of one or more Bacillus or Lactobacillus strain have capability and activity to reduce methane gas production and/or with capability and activity to inhibit methanogenic Archaea and/or bacteria or to otherwise alter the environment or microbiome including methanogenic Archaea and/or bacteria to reduce methane production and alter methanogenesis. In some embodiments, combinations of two or more Bacillus strain have capability and activity to reduce methane gas production and/or with capability and activity with regard to methanogenic Archaea and/or bacteria. In some embodiments, combinations of one or more Bacillus strain and a Lactobacillus strain have capability and activity to reduce methane gas production and/or with capability and activity with regard to methanogenic Archaea and/or bacteria.

In an embodiment, Bacillus amyloliquefaciens strain BE191006 (also denoted as ELA191006) corresponding to ATCC deposit PTA-127065 is provided. In an embodiment, Bacillus amyloliquefaciens strain BE202071 (also denoted as ELA202071) corresponding to ATCC deposit PTA-127064 is provided. In an embodiment, Bacillus subtilis strain BE191105 (also denoted as ELA191105) corresponding to ATCC deposit PTA-126786 is provided.

Compositions of one or more Bacillus strain are provided for reducing methane gas production and/or altering methanogenesis and/or inhibiting or altering the microbiome including methanogenic Archaea. In some embodiments, a composition includes or comprises isolated Bacillus amyloliquefaciens strain BE191006. In some embodiments, a composition includes or comprises isolated Bacillus amyloliquefaciens strain BE202071. In some embodiments, a composition includes or comprises isolated Bacillus subtilis strain including BE191105. In some embodiments, a composition includes a first isolated Bacillus amyloliquefaciens strain BE191006 and a second isolated Bacillus amyloliquefaciens strain BE202071. In some embodiments, a composition includes a first isolated Bacillus amyloliquefaciens strain BE191006 and isolated Bacillus subtilis strain BE191105. In some embodiments, the composition includes a first isolated Bacillus amyloliquefaciens strain BE191006, a second isolated Bacillus amyloliquefaciens strain including BE202071, and a first isolated Bacillus subtilis strain BE191105.

In some embodiments, the isolated Bacillus subtilis strain BE191105 is genetically modified. In some embodiments, the isolated Bacillus subtilis strain BE191105 is genetically modified to produce or deliver an antibacterial agent, antibacterial peptide or anti-methanogen agent or compound. In some instances, the bacterial strains are combined in a composition or are combined upon delivery or administration with one or more antibacterial agent, antibacterial peptide or anti-methanogen agent or compound. Antibacterial peptides may include for example CAP 18 peptides. CAP 18 peptides arc described in PCT/US2022/044221, filed Sep. 21, 2022, incorporated by reference herein.

In an embodiment of the invention, a probiotic composition or microbiome modulator or direct feed microbial is provided which comprises at least one Bacillus strain, wherein the Bacillus strain is capable of reducing methane gas production and/or inhibiting or altering the microbiome including methanogenic Archaea and/or bacteria. In an embodiment of the invention, a probiotic composition or microbiome modulator or direct feed microbial is provided which comprises at least one Lactobacillus strain, wherein the Lactobacillus strain is capable of reducing methane gas production and/or inhibiting or altering the microbiome including methanogenic Archaea and/or bacteria. In an embodiment of the invention, a probiotic composition or microbiome modulator or direct feed microbial is provided which comprises at least one Lactobacillus strain and at least one Bacillus strain, wherein the Lactobacillus strain and the Bacillus strain are capable of reducing methane gas production and/or inhibiting or altering the microbiome including methanogenic Archaea and/or bacteria.

In an embodiment of the invention, a probiotic composition or microbiome modulator or methane production modulator capable of reducing methane gas production and/or inhibiting or otherwise altering the microbiome including methanogenic Archaea and/or bacteria is provided which comprises at least one Bacillus strain. In an embodiment of the invention, a probiotic composition or microbiome modulator or direct feed microbial capable of reducing methane gas production and/or inhibiting or otherwise altering the microbiome including methanogenic Archaea and/or bacteria is provided which comprises a combination of at least two Bacillus strains. In embodiments, the Bacillus strain or strains are selected from Bacillus strain 06 (BE191006) or a Bacillus strain having at least 90% identity, 95% identity, 97% identity, 98% identity, 99% identity in genomic sequence to the sequence of Strain 06 (BE191006); Strain 71 (BE202071) or a Bacillus strain having at least 90% identity, 95% identity, 97% identity, 98% identity, 99% identity in genomic sequence to the sequence of Strain 71 (BE202071); and Strain 105 (BE191105) or a Bacillus strain having at least 90% identity, 95% identity, 97% identity, 98% identity, 99% identity in genomic sequence to the sequence of and Strain 105 (BE191105).

In an embodiment of the invention, a probiotic composition or microbiome modulator or direct feed microbial capable of reducing methane gas production and/or inhibiting methanogenesis via methanogenic Archaea is provided which comprises at least one Bacillus strain. In an embodiment of the invention, a probiotic composition or microbiome modulator or direct feed microbial capable of reducing methane gas production and/or inhibiting methanogenesis via methanogenic Archaea is provided which comprises a combination of at least two Bacillus strains. In embodiments, the Bacillus strain or strains are selected from BE191006 or a Bacillus strain having at least 90% identity, 95% identity, 97% identity, 98% identity, 99% identity in genomic sequence to the sequence of SEQ ID NO: 27; BE202071 or a Bacillus strain having at least 90% identity, 95% identity, 97% identity, 98% identity, 99% identity in genomic sequence to the sequence of SEQ ID NO: 42; and BE191105 or a Bacillus strain having at least 90% identity, 95% identity, 97% identity, 98% identity, 99% identity in genomic sequence to one or more of SEQ ID NO: 1, 2, 3, 4 and 5.

In some embodiments, the invention relates to related, homologous or derivative Bacillus strains having significant genome sequence identity to the genome sequence of any of Bacillus amyloliquefaciens strain BE191006 (also denoted ELA191006) corresponding to ATCC deposit PTA-127065, Bacillus amyloliquefaciens strain BE202071 (also denoted ELA202071) corresponding to ATCC deposit PTA-127064, and/or Bacillus subtilis strain BE191105 (also denoted ELA191105) corresponding to ATCC deposit PTA-126786. Thus, derivative or similar or nearly genetically identical strains to the Bacillus strains provided herein are contemplated by and additional embodiments of the invention. Bacillus strains having 80% identity, 85% identity, 90% identity, 95% identity, 97% identity, 98% identity, 99% identity in genomic sequence to a strain provided and deposited in association with this invention are contemplated and are embodiments of the invention. Such derivative or similar or nearly genetically identical strains must similarly function as probiotics and have activity/capability or function in improving animal health and animal production and performance, including as detailed in the capability and activity or function of the strains and examples hereof. Exemplary of this embodiment, it is noted that Bacillus amyloliquefaciens strain BE191024 corresponding to ATCC deposit PTA-126784 and Bacillus amyloliquefaciens strain BE191006 corresponding to ATCC deposit PTA-127065 are genetically related or similar strains, demonstrating 99% identity in genome sequence.

In embodiments, the Bacillus strains are selected from BE191006 corresponding to ATCC deposit PTA-127065 or a Bacillus strain having at least 90% identity, 95% identity, 97% identity, 98% identity, 99% identity in genomic sequence to the sequence of BE191006 corresponding to ATCC deposit PTA-127065; BE202071 corresponding to ATCC deposit PTA-127064 or a Bacillus strain having at least 90% identity, 95% identity, 97% identity, 98% identity, 99% identity in genomic sequence to the sequence of BE202071 corresponding to ATCC deposit PTA-127064; and BE191105 corresponding to ATCC deposit PTA-126786 or a Bacillus strain having at least 90% identity, 95% identity, 97% identity, 98% identity, 99% identity in genomic sequence to the sequence of BE191105 corresponding to ATCC deposit PTA-126786.

In accordance with one embodiment of the invention, a probiotic composition or microbiome modulator capable of reducing methane gas production and/or inhibiting or altering the microbiome of methanogenic Archaea and/or bacteria is provided comprising one or more Bacillus strain, particularly one or more Bacillus subtilis strain or Bacillus amyloliquefaciens strain. In accordance with one embodiment of the invention, a probiotic composition or microbiome modulator capable of reducing methane gas production and/or inhibiting or altering the microbiome including methanogenic Archaea is provided comprising at least one of: a first isolated Bacillus amyloliquefaciens strain, a second isolated Bacillus amyloliquefaciens strain, and a first isolated Bacillus subtilis strain; and a carrier suitable for animal administration; wherein said composition reduces methane production and/or reduces or inhibits the colonization of an animal by a methanogenic bacteria or bacterium when an effective amount is administered to an animal, as compared to an animal not administered the composition. In accordance with one embodiment of the invention, a probiotic composition or microbiome modulator capable of reducing methane gas production and/or inhibiting or altering the microbiome including methanogenic Archaea and/or bacteria is provided comprising at least one of: a first isolated Bacillus amyloliquefaciens strain, a second isolated Bacillus amyloliquefaciens strain, and a first isolated Bacillus subtilis strain; and a carrier suitable for animal administration; wherein said composition reduces methane production and/or reduces or inhibits the colonization of or methanogenesis in an animal by a methanogenic Archaea when an effective amount is administered to an animal, as compared to an animal not administered the composition; and wherein the first isolated Bacillus amyloliquefaciens strain comprises a nucleic acid sequence having at least 95%, at least 96%, at least 97%, at least 98%, or at least 99% sequence identity with SEQ ID NO: 27, and/or with nucleic acid encoding one or more protein of SEQ ID NO: 28-41; wherein the second Bacillus amyloliquefaciens strain comprises a nucleic acid sequence having at least 95%, at least 96%, at least 97%, at least 98%, or at least 99% sequence identity with SEQ ID NO: 42, and/or with nucleic acid encoding one or more protein of SEQ ID NO: 43-50; wherein the first Bacillus subtilis strain comprises a nucleic acid sequence having at least 95%, at least 96%, at least 97%, at least 98%, or at least 99% sequence identity with SEQ ID NO: 1, 2, 3, 4 and/or 5, or with a nucleic acid sequence encoding one or more protein of SEQ ID NO: 6-26.

In an embodiment, the composition comprises at least two bacterial strains. In an embodiment, the composition comprises at least two bacterial strains capable of reducing methane production in an animal and/or reducing or inhibiting the colonization of or the microbiome in an animal including methanogenic Archaea and/or bacteria. In an embodiment, the composition comprises at least two bacterial strains capable of reducing methane production in a ruminant animal and/or reducing or inhibiting the colonization of or altering the microbiome in a ruminant animal including methanogenic Archaea. In an embodiment, the composition comprises at least two Bacillus strains. In an embodiment, the composition comprises a Bacillus strain and a Lactobacillus strain. In an embodiment, the composition comprises at least two of: a first isolated Bacillus amyloliquefaciens strain, a second isolated Bacillus amyloliquefaciens strain, and an isolated Bacillus subtilis strain. In an embodiment, the composition comprises a first isolated Bacillus amyloliquefaciens strain, a second isolated Bacillus amyloliquefaciens strain, and an isolated Bacillus subtilis strain. In an embodiment, the composition comprises at least two of: a first isolated Bacillus amyloliquefaciens strain, a second isolated Bacillus amyloliquefaciens strain, an isolated Bacillus subtilis strain, and an isolated Lactobacillus reuteri strain. In an embodiment, the composition comprises a first isolated Bacillus amyloliquefaciens strain or a second isolated Bacillus amyloliquefaciens strain, an isolated Bacillus subtilis strain, and an isolated Lactobacillus reuteri strain.

In embodiments, the composition comprises at least two isolated strains capable of inhibiting methane productions or methanogenesis and selected from one or more Bacillus amyloliquefaciens strain, a Bacillus subtilis strain, and a Lactobacillus reuteri strain. In an embodiment, a composition is provided comprising at least two strains selected from Bacillus amyloliquefaciens strain BE191024, Bacillus amyloliquefaciens strain BE191006, Bacillus subtilis strain BE191105, and Lactobacillus reuteri strain 3632. Compositions comprising Bacillus amyloliquefaciens strain BE191024 and Bacillus amyloliquefaciens strain BE191006 are one embodiment. Compositions comprising Bacillus amyloliquefaciens strain BE191024 and Bacillus subtilis strain BE191105 are one embodiment. Compositions comprising Bacillus amyloliquefaciens strain BE191006 and Bacillus subtilis strain BE191105 are one embodiment. Compositions comprising Bacillus amyloliquefaciens strain BE191024 and Bacillus subtilis strain BE191105 are one embodiment. Compositions comprising Bacillus amyloliquefaciens strain BE191024 and Lactobacillus reuteri strain 3632 are one embodiment. Compositions comprising Bacillus amyloliquefaciens strain BE191006 and Lactobacillus reuteri strain 3632 are one embodiment. Compositions comprising Bacillus subtilis strain BE191105 and Lactobacillus reuteri strain 3632 are one embodiment. Compositions comprising Bacillus amyloliquefaciens strain BE191024 or Bacillus amyloliquefaciens strain BE191006, Bacillus subtilis strain BE191105 and Lactobacillus reuteri strain 3632 are one embodiment. Compositions comprising Bacillus amyloliquefaciens strain BE191024, Bacillus amyloliquefaciens strain BE191006, Bacillus subtilis strain BE191105 and Lactobacillus reuteri strain 3632 are one embodiment.

In an embodiment, the composition comprises at least two of a Bacillus amyloliquefaciens strain, a Bacillus subtilis strain and a Lactobacillus strain. In an embodiment, the composition comprises at least two of a Bacillus amyloliquefaciens strain, a Bacillus subtilis strain and a Lactobacillus reuteri strain. In an embodiment the isolated Lactobacillus reuteri strain corresponds to Lactobacillus reuteri strain 3632, which corresponds to ATCC Patent Deposit Number PTA-126788, or a Lactobacillus reuteri strain having at least 90% identity, 95% identity, 97% identity, 98% identity, 99% identity in genomic sequence to the sequence of Lactobacillus reuteri strain 3632 corresponding to ATCC deposit PTA-126788. In one embodiment, the isolated Lactobacillus reuteri strain has a genomic nucleic acid sequence including at least one of SEQ ID NOs: 51-57, sequences having one or more nucleic acid sequence difference from the sequence of at least one of SEQ ID NOs: 51-57, sequences having at least one nucleic acid sequence difference from the sequence of at least one of SEQ ID NOs: 51-57 and further having at least 97%, at least 98%, at least 99% or at least 99.5% sequence identity with one or more of SEQ ID NOs: 51-57.

In an embodiment, the composition includes a carrier. In an embodiment of the composition, the carrier is selected from edible food grade material, mineral mixture, gelatin, cellulose, carbohydrate, starch, glycerin, water, rice hulls, glycol, molasses, calcium carbonate, whey, sucrose, dextrose, soybean oil, vegetable oil, sesame oil, and corn oil.

In an embodiment, the composition includes one or more anti-methanogenic compound. In an embodiment, the composition is administered or fed in combination with one or more anti-methanogenic compound. A compound is determined to be anti-methanogenic based on its mechanism. In an embodiment, the composition includes one or more compound capable of reducing or inhibiting methane (CH4) production, release, or stability. In an embodiment, the composition is administered or fed in combination with one or more compound capable of reducing or inhibiting methane (CH4) production, release, or stability. Exemplary anti-methanogenic compounds or inhibitors of methane include inhibitors of a rate-limiting enzyme in methanogenesis pathway, such as the enzyme methyl Coenzyme M reductase. Exemplary such inhibitors include 3-NOP, bromoethanesulfonate, bromoform, red algae/Asparagopsis taxiformis. Other exemplary anti-methanogenic compounds or inhibitors of methane include inhibitors of methanogens lipid biosynthesis, such as the inhibitor mevinolin. Further other anti-methanogenic compounds or inhibitors of methane include microbiome modulators, such as monensin. Thus, compositions of one or more strains provided herein with one or more anti-methanogenic compound selected from inhibitors of a rate-limiting enzyme in methanogenesis pathway, inhibitors of methanogens lipid biosynthesis, or microbiome modulators are contemplated and provided herein.

In an embodiment, the composition does not comprise Lactobacillus. In an embodiment, the composition does not comprise non-Bacillus strains. In an embodiment, Bacillus amyloliquefaciens and/or Bacillus subtilis are the only bacterial strains in the composition. In an embodiment, Lactobacillus reuteri and a Bacillus strain are the only bacterial strains in the composition.

The genome nucleic acid sequence of Bacillus amyloliquefaciens strain BE191006 (also denoted BAMY 19006 or BAMY 006) is provided in sequence SEQ ID NO: 27. The genome nucleic acid sequence of Bacillus amyloliquefaciens strain BE202071 (also denoted ELA202071, BAMY 202071 or BAMY 071) is provided in sequences SEQ ID NO: 42. The genome nucleic acid sequence of Bacillus subtilis strain BE191105 (also denoted ELA1901105 and BSUB 19105 and BSUB 105) is provided in sequences SEQ ID NO:s 1, 2, 3, 4 and/or 5. Genomically related or variant Bacillus amyloliquefaciens strains having at least 80%, at least 85%, at least 90% at least 95%, at least 97%, at least 98%, at least 99% nucleic acid sequence identity to the genome sequence of SEQ ID NO: 27 or of SEQ ID NO: 42 are provided and contemplated as embodiments of the invention. Genomically related or variant Bacillus subtilis strains having at least 80%, at least 85%, at least 90% at least 95%, at least 97%, at least 98%, at least 99% nucleic acid sequence identity to the genome sequence of SEQ ID NO: 1, 2, 3, 4 and/or 5 are provided and contemplated as embodiments of the invention.

In one embodiment, the isolated Bacillus strain has a genomic nucleic acid sequence including at least one of SEQ ID NOs: 1, 2, 3, 4, or 5, sequences having one or more nucleic acid sequence difference from the sequence of at least one of SEQ ID NOs: 1, 2, 3, 4, or 5, sequences having at least one nucleic acid sequence difference from the sequence of at least one of SEQ ID NOs: 1, 2, 3, 4, or 5 and further having at least 97%, at least 98%, at least 99% or at least 99.5% sequence identity with one or more of SEQ ID NOs: 1, 2, 3, 4 or 5. In one embodiment, the isolated Bacillus strain has a genomic nucleic acid sequence including at least one of SEQ ID NO: 27, sequences having one or more nucleic acid sequence difference from the sequence of at least one of SEQ ID NO: 27, sequences having at least one nucleic acid sequence difference from the sequence of at least one of SEQ ID NO: 27 and further having at least 97%, at least 98%, at least 99% or at least 99.5% sequence identity with one or more of SEQ ID NO: 27. In one embodiment, the isolated Bacillus strain has a genomic nucleic acid sequence including at least one of SEQ ID NO: 42, sequences having one or more nucleic acid sequence difference from the sequence of at least one of SEQ ID NO: 42, sequences having at least one nucleic acid sequence difference from the sequence of at least one of SEQ ID NO: 42 and further having at least 97%, at least 98%, at least 99% or at least 99.5% sequence identity with one or more of SEQ ID NO: 42.

Such genomically related or variant Bacillus strains are capable of reducing methane production in an animal and/or reducing or inhibiting the colonization of or the microbiome in an animal including methanogenic Archaea and/or bacteria. Such genomically related or variant Bacillus strains are comparably capable of improving animal health and animal production performance. Such genomically related or variant bacillus strains are capable of use and application in compositions in accordance with the invention. In an embodiment, such genomically related sequences include nucleic acid encoding one or more proteins provided herein as unusual genes or proteins of the respective strains. For example and illustratively, such proteins include SEQ ID NOs: 28-41 for strain BAMY 006, include proteins SEQ ID NOs: 43-50 for strain BAMY 071, and include proteins SEQ ID NOs: 6-26 for strain BSUB 105.

In embodiments, a feed additive of the probiotic composition or microbiome modulator is provided. In an embodiment, the feed additive comprises a combination of the spore forms of at least two of the Bacillus strains provided herein. In an embodiment, the feed additive comprises the spore forms of one or more of the Bacillus strains provided herein and further comprises the Lactobacillus strain provided herein. In an embodiment, the feed additive comprises one or more of the Bacillus strains provided herein and further comprises the Lactobacillus strain provided herein.

In some embodiments, particularly wherein more than one Bacillus and/or Lactobacillus is included in the composition, the ratio of strains is about 0.75-1.5:1. In some embodiments, the ratio of a first isolated Bacillus amyloliquefaciens strain, second isolated Bacillus amyloliquefaciens strain, and/or the isolated Bacillus subtilis strain is about 0.75-1.5:1:0.75-1.5. In some embodiments, the ratio of a first and second isolated Bacillus strain and the isolated Bacillus subtilis strain is about 0.75-1.5:1:0.75-1.5. In some embodiments, the ratio of a first and second isolated Bacillus strain and the isolated Lactobacillus reuteri strain is about 0.75-1.5:1:0.75-1.5. In some embodiments, the ratio of a first Bacillus strain and the isolated Lactobacillus strain is about 0.75-1.5:1. In some embodiments, the ratio of a first Bacillus strain and the isolated Lactobacillus reuteri strain is about 0.75-1.5:1. In a preferred embodiment, the composition contains equal amounts of the strains disclosed herein and above. The amount or ratio can be determined or characterized by any known method. For example, the ratio or amount can be characterized by the number of viable spores per gram dry weight of the probiotic composition.

In some embodiment, bacterial strains of the present disclosure capable of reducing methane production in an animal and/or reducing or inhibiting the colonization of an animal by a methanogenic bacteria include those that comprise polypeptide sequences that share at least 70%, 75%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity with at least one of: SEQ ID NOs: 28, 29, 30, 31, 32, 33, 34, 35, 36, 37, 38, 39, 40 and 41. In a further embodiment, bacterial strains of the present disclosure capable of reducing methane production in an animal and/or reducing or inhibiting the colonization of an animal by a methanogenic bacteria include those that comprise polypeptide sequences that share at least 70%, 75%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity with at least one of: SEQ ID NOs: 43, 44, 45, 46, 47, 48, 49 and 50. In a further embodiment, bacterial strains of the present disclosure capable of reducing methane production in an animal and/or reducing or inhibiting the colonization of an animal by a methanogenic bacteria include those that comprise polynucleotide sequences that encodes for a polypeptide sequence that share at least 70%, 75%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity with at least one of: SEQ ID NOs: 6, 7, 8, 9, 10, 11, 12, 13, 14, 15, 16, 17, 18, 19, 20, 21, 22, 23, 24, 25 and 26.

In accordance with one embodiment of the invention, a probiotic composition or microbiome modulator capable of reducing methane gas production and/or inhibiting or altering the microbiome of methanogenic Archaea and/or bacteria is provided comprising a Lactobacillus strain. In an embodiment, a probiotic composition or microbiome modulator capable of reducing methane gas production and/or inhibiting or altering the microbiome of methanogenic Archaea and/or c bacteria is provided comprising a Lactobacillus reuteri strain.

In accordance with one embodiment of the invention, a probiotic composition or microbiome modulator capable of reducing methane gas production is provided comprising at least an isolated Lactobacillus strain; and a carrier suitable for animal administration; wherein said composition reduces methane production and/or reduces or inhibits the colonization of or alters the microbiome in an animal including methanogenic Archaea when an effective amount is administered to an animal, as compared to an animal not administered the composition. In an embodiment, the probiotic composition or microbiome modulator comprises at least an isolated Lactobacillus reuteri strain; and a carrier suitable for animal administration; wherein said composition reduces methane production and/or reduces or inhibits the colonization of or alters the microbiome in an animal including methanogenic Archaea and/or bacteria when an effective amount is administered to an animal, as compared to an animal not administered the composition. In an embodiment the isolated Lactobacillus reuteri strain corresponds to Lactobacillus reuteri strain 3632, which corresponds to ATCC Patent Deposit Number PTA-126788, or a Lactobacillus reuteri strain having at least 90% identity, 95% identity, 97% identity, 98% identity, 99% identity in genomic sequence to the sequence of Lactobacillus reuteri strain 3632 corresponding to ATCC deposit PTA-126788.

In one embodiment, the isolated Lactobacillus reuteri strain has a genomic nucleic acid sequence including at least one of SEQ ID NOs: 51-57, sequences having one or more nucleic acid sequence difference from the sequence of at least one of SEQ ID NOs: 51-57, sequences having at least one nucleic acid sequence difference from the sequence of at least one of SEQ ID NOs: 51-57 and further having at least 97%, at least 98%, at least 99% or at least 99.5% sequence identity with one or more of SEQ ID NOs: 51-57.

In an embodiment, the isolated Lactobacillus reuteri strain is Lactobacillus reuteri strain 3632, which corresponds to ATCC Patent Deposit Number PTA-126788. In one embodiment, the isolated Lactobacillus reuteri strain comprises or has a genomic nucleic acid sequence corresponding to the genomic nucleic acid sequence of ATCC strain PTA-126788, or a variant thereof comprising or having a nucleic acid sequence at least 98%, at least 98.5%, at least 99%, or at least 99.5% sequence identity to the genomic nucleic acid sequence of ATCC strain PTA-126788.

In an embodiment, a method is provided for reducing methane emission or production by methanogenic organisms such as methanogenic Archaea or bacteria in the environment, in a culture or in a process comprising introducing or otherwise contacting the composition hereof or a fermentation product of the composition. In one embodiment, the environment, culture or process is selected from manure, litter, ponds, an aerobic digester, and waste treatment.

In embodiments, methods are provided for reducing methane emission or production, in instances by methanogenic organisms, in manure by combining or adding to the manure one or more compositions hereof or fermentation product of one or more compositions hereof.

In another embodiment, a postbiotic is provided comprising the fermentation product, culture or supernatant of the composition of strains hereof. In an embodiment, the postbiotic reduces methane production, alters methanogenesis, and/or reduces greenhouse emissions. In an embodiment, the postbiotic is administered to an animal.

While there have been described what are presently believed to be the preferred embodiments of the present invention, those skilled in the art will realize that other and further changes and modifications may be made thereto without departing from the spirit of the invention, and it is intended to claim all such modifications and changes as come within the true scope of the invention.

Other objects and advantages will become apparent to those skilled in the art from a review of the ensuing detailed description, which proceeds with reference to the following illustrative drawings, and the attendant claims.

### BRIEF DESCRIPTION OF DRAWINGS

FIG. 1 depicts methane emission by enteric fermentation.

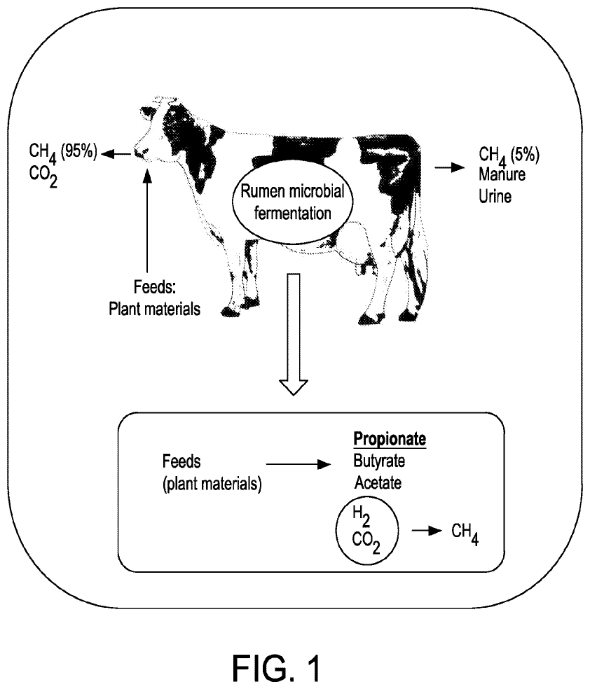

FIG. 2 depicts enteric methane emission.

FIGS. 3A and B. (A) depicts methane production with an H₂ consuming and H₂ producing path being shown. (B) depicts alternative H₂ sinks, with it being shown how H₂ flow can be redirected to an alternative electron acceptor (such as SO₄²⁻ and NO₃⁻) or pathway to reduce methane.

FIG. 4 depicts the methanogenesis pathway (Wolfe cycle).

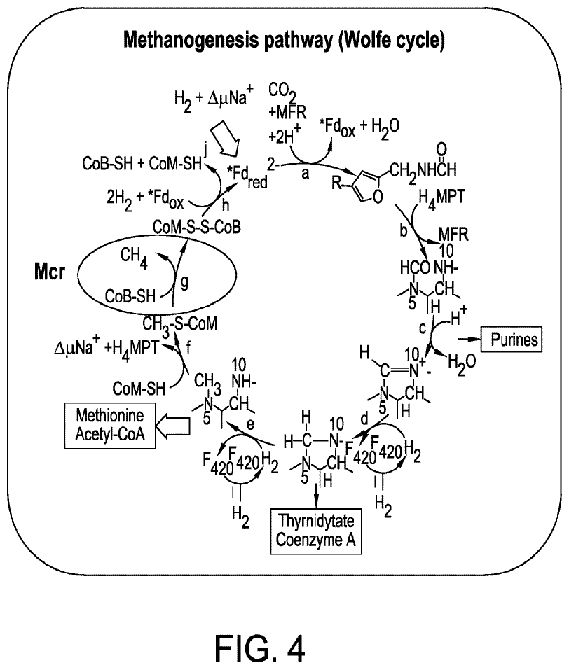

FIG. 5 depicts the experimental design of the rumen batch culture and methane assessment.

FIG. 6 depicts a methane standard curve representing the amount of methane gas injected into the gas chromatography (GC) system.

FIG. 7 depicts results with a control series, the control negative being CaCO₃ and the control positive being CHCl₃. Change in pressure (AP) in psig and change in concentration of methane (ΔConcentration) in mM are graphed over time in hours (h).

FIG. 8 depicts results with a Bacillus bacteria series.

FIG. 9 depicts results with a Lactobacillus bacteria series.

FIG. 10 depicts a comparison result of Bacillus and Lactobacillus strains versus control.

FIG. 11 depicts methane production (CH₄ production, %) for various additives and bacteria strains tested. Methane production is defined as the increase (+ value) or decrease (− value) of methane production of a typical treatment when compared to a control blank (None).

FIGS. 12A and B provides results of methane production assessment in rumen batch culture. The results are depicted in alternative graph formats. In (A) methane production with addition of each of None (nothing added), CHCl3, Vehicle, bacteria strain B. subtilis BE105, B. amyloliquefaciens BE006, B. amyloliquefaciens BE071, L. reuteri BE3632, another strain denoted Strain A is shown on a scale of 0 to 100. In (B) methane production with addition of each of None (nothing added), CHCl3, Vehicle, bacteria strain B. subtilis BE105, B. amyloliquefaciens BE006, B. amyloliquefaciens BE071, L. reuteri BE3632, and another strain Strain A is shown on a scale of 0 to 125. Bacillus amyloliquefaciens strain BE006 is noted as reducing methane production by at least 25%.

FIG. 13 depicts the design of an in vivo in animal study to further evaluate the effect of administration of bacteria capable of reducing methane production in cattle performance.

FIG. 14 depicts the effect of L. reuteri BE3632 addition on H₂, CO₂ and CH₄ in in vitro rumen batch culture. The left panel shows H₂, the center panel shows CO₂ and the right panel shows CH₄. Sampling of untreated, CHCl₃ treated, media alone, and bacteria L. reuteri BE3632 is provided. Assessments were conducted at 0, 24 and 48 hours after treatment or addition.

FIG. 15 provides a diagram of the process steps to evaluate L. reuteri BE3632 colonization in in vitro rumen batch culture. Starting with an original inoculum which was sampled and plated as Plate 1, an inoculum was transferred in series as a 1^{st} transfer through a 3^{rd} transfer with an inoculum from each plated as Plate 2 through Plate 4, with each transfer occurring after 2 days. The bacteria present in Plate 1 to Plate 4 were counted.

FIG. 16 shows a sample of plate evaluations with bacteria from the study described in FIG. 15. Bacterial colonies of L. reuteri BE3632 are evident (orange colonies).

FIG. 17 depicts an overlay experiment to examine strain compatibility. Evaluation of strains B. subtilis BE105 and L. reuteri BE3632 is depicted. The steps and process for others strains would be the same.

### DETAILED DESCRIPTION OF THE INVENTION

The present disclosure provides a solution to the problem of greenhouse gas emissions, including to the problem of methane emission from various sources or under various conditions. The present disclosure provides a solution to the problem of greenhouse gas emissions, including to the problem of methane emission from animals including livestock, such as ruminants, thereby improving livestock production sustainability and ruminal feed efficiency. Specifically, the present disclosure provides for the development of a feed additive for reduction of enteric methane gas emissions from animals including livestock, including or particularly ruminants, to reduce the carbon footprint from livestock production, to provide for manure management, and to provide for ruminal feed efficiency.

Sustainability of livestock ruminant production and improvement of feed efficiency are thus advantages of the present invention. In particular, reduced methane leads to increased feed efficiency which leads to sustainable livestock production. A direct effect on reduction of enteric methane emission is contemplated. A direct effect on reduction of enteric methane emission, wherein methane emission or production is reduced by at least 10%, at least 15%, at least 20%, at least 25%, at least 30%, at least 35%, at least 40%, at least 45%, at least 50% is contemplated. A direct effect on reduction of enteric methane emission, wherein methane emission or production is reduced by at least 10% is contemplated. A direct effect on reduction of enteric methane emission, wherein methane emission or production is reduced by at least 20% is contemplated. A direct effect on reduction of enteric methane emission, wherein methane emission or production is reduced by at least 25% is contemplated.

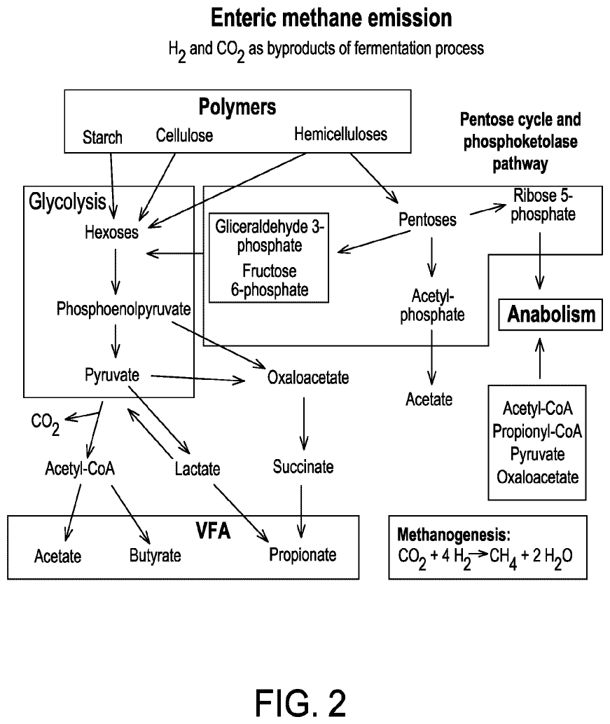

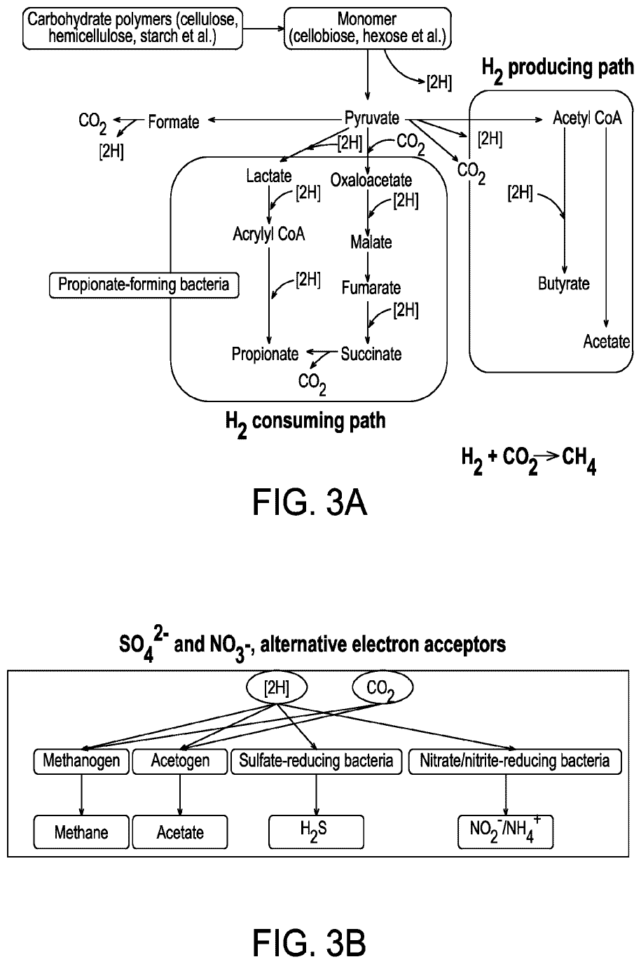

The present disclosure provides a mitigation strategy for enteric methane production. Specifically, the present disclosure provides for the use of probiotics or microbiome modulators to mitigate enteric methane production in animals, such as livestock and, more particularly, ruminants. Also, the present disclosure provides for the use of probiotics or microbiome modulators to mitigate methane production by methanogenic Archaea or in situations or locations where methanogenic Archaea are present, including in animals, in manure, deep in or at the bottom of ponds, in anaerobic digesters, etc. Probiotics can have an effect on methanogenesis through modulation of rumen microbiota. Without wishing to be bound by any particular theory, by modulating microbiome structure, the probiotics or microbiome modulators decrease bacteria that produce H₂ available for methanogenesis, thereby decreasing the amount of methanogenesis with the effect of reducing methane emissions. The probiotics or microbiome modulators amplify or increase the H₂ consuming path and reduce the H₂ producing path, thereby reducing the H₂ available to generate methane CH4 (see for example FIG. 3A). Production of volatile fatty acid (VFA) with the fermentation process can thereby be increased as opposed to methanogenesis (see for example FIG. 2).

The present disclosure provides probiotics or microbiome modulators that alter or target methanogens in the gastrointestinal tract of an animal. In particular, the probiotics of the present disclosure alter the fermentation process of methanogens in the rumen and, as such, are rumen modifiers. In particular, the probiotics of the present disclosure alter the fermentation process of methanogen Archaea in the rumen and, as such, are rumen modifiers. In particular, the probiotics or microbiome modulators of the present disclosure alter the microbiome and thereby the fermentation process and methanogen and CH₄ production. Methanogens which can be targets of the probiotics of the present disclosure include, without limitation, Methanobrevibacter ruminantium, Methanosphaera stadtmanae, Methanomicrobium mobile, Methanomassilicoccaceae spp., Methanobacterium bryantii and Methanosarcina mazei.

Methanogens are a group of microorganisms that produce methane as a byproduct of their metabolism. They play an important role in the digestive system of ruminants. The stomach of ruminants contains four major parts: rumen, reticulum, omasum and abomasum. The food with saliva first passes to the rumen for breaking into smaller particles and then moves to the reticulum, where the food is broken into further smaller particles. Any indigestible particles are sent back to the rumen for rechewing. The majority of anaerobic microbes assisting the cellulose breakdown occupy the rumen and initiate the fermentation process. The animal absorbs the fatty acids, vitamins and nutrient content on passing the partially digested food from the rumen to the omasum. This decreases the pH level and initiates the release of enzymes for further breakdown of the food which later passes to the abomasum to absorb remaining nutrients before excretion. This process takes about 9-12 hours.

Some of the microbes in the ruminant digestive system are: Fibrobacter (Bacteroides) succinogenes is a gram negative, cellulolytic and amylolytic methanogen that produces formates, acetates and succinates; Ruminococcus albus is a cellulolytic, xylanolytic bacterium producing ethanol, hydrogen, carbon dioxide, formates and acetates; Ruminococcus flavefaciens is a cellulolytic, xylanolytic bacteria producing formates, acetates, hydrogen and succinates; Butyrivibrio fibrisolvens [is a proteolytic, cellulolytic, xylanolytic microbe producing lactate, butyrate, ethanol, hydrogen, carbon dioxide, formates and acetates; Streptococcus bovis is an amylolytic, major soluble sugar fermenter, proteolytic, microbe resulting in lactate, acetate and formate; Ruminobacter (Bacteroides) amylophilus amylolytic, propionate, proteolytic, organism that forms, formates, acetates and succinates; Prevotella (Bacteroides) ruminocola amylolytic, xylanolytic, propionate, proteolytic, microbe that creates, formates, acetates, succinates and propionate; Succinimonas amylolytica amylolytic, dextrinolytic, bacteria forming acetates and succinates; Selenomonas ruminantium amylolytic, major soluble sugar fermenter, glycerol-utilizing, lactate-utilizing, proteolytic, microbe producing acetates, lactates, hydrogen, carbon dioxide and propionates; Lachnospira multiparus propionate, proteolytic, A microbe that results in production of lactate, ethanol, hydrogen, carbon dioxide, formates and acetates; Succinivibrio dextrinosolvens propionate, dextrinolytic, bacteria forming formates, acetates, lactates and succinates; Methanobrevibacter ruminantium methanogenic, hydrogen utilizing, Archaea involved in the creation of methane; Methanosarcina barkeri methanogenic, hydrogen utilizing, Archaea involved in the creation of methane.

Ruminants (suborder Ruminantia) are hoofed herbivorous grazing or browsing mammals that are able to acquire nutrients from plant-based food by fermenting it in a specialized stomach prior to digestion, principally through microbial actions. The process, which takes place in the front part of the digestive system and therefore is called foregut fermentation, typically requires the fermented ingesta (known as cud) to be regurgitated and chewed again. The process of rechewing the cud to further break down plant matter and stimulate digestion is called rumination. The roughly 200 species of ruminants include both domestic and wild species. Ruminating mammals include cattle, all domesticated and wild bovines such as cattle, bison, American buffalo and water buffalos, goats, sheep, giraffes, deer, gazelles, and antelopes. Ruminants play an important role in global food security and nutrition, as well as in the livelihoods of farmers and those along the agrifood chain. Ruminant animals include cattle, sheep, goats and buffalo and are the largest livestock production system in the world. Over 50 percent of all protein supplied from the livestock sector comes from ruminants, mainly in the form of milk and meat. Globally, enteric methane emissions from ruminants and manure management practices account for over 30 percent of all human-induced methane emissions. Cattle account for 77 percent of methane emissions, buffalo for 14 percent and small ruminants, such as sheep and goats for the remainder.

Methane emissions from non-ruminant animals are also relevant and contribute to green house gas (GHG). In some countries the emission contribution from non-ruminants is significant. For instance in East and Southeast Asia, swine and poultry account for much of the enteric methane. Methanogens are also associated with some clinically relevant conditions in animals, including humans. Methanogens and methane production in humans are positively correlated with obesity, higher glucose levels and greater body mass index (Mathur R et al (2013) Obesity 21 (4): 748-754; Mathur R et al (2013) Clin Endocrinol Metab 98 (4): E698-702; Mathur R et al (2014) Res J or Endocrinol and Metab doi:10.7243/2053-3640-2-2).

Vertebrates lack the ability to hydrolyze the beta [1-4] glycosidic bond of plant cellulose due to the lack of the enzyme cellulase. Thus, ruminants completely depend on the microbial flora, present in the rumen or hindgut, to digest cellulose. Digestion of food in the rumen is primarily carried out by the rumen microflora, which contains dense populations of several species of bacteria, protozoa, sometimes yeasts and other fungi—1 ml of rumen is estimated to contain 10-50 billion bacteria and 1 million protozoa, as well as several yeasts and fungi.

Since the environment inside a rumen is anaerobic, most of these microbial species are obligate or facultative anaerobes that can decompose complex plant material, such as cellulose, hemicellulose, starch, and proteins. The hydrolysis of cellulose results in sugars, which are further fermented to acetate, lactate, propionate, butyrate, carbon dioxide, and methane.

The compositions and methods provided herein have application in non-ruminant animals, including humans. Human food consumption leads to flatulence and methane release, which contributes to greenhouse gases. There are some intestinal conditions in humans associated with increased methane production. Small intestinal bacterial overgrowth (SIBO) is a condition associated with large numbers of bacteria colonizing the small intestine (Rezaie A, Pimentel M, Rao S S. Curr Gastroenterol Rep. 2016; 18 (2):8; doi.org/10.1007/s11894-015-0482-9). Intestinal methanogen overgrowth (IMO) is a newer term (possibly replacing methane dominant SIBO) to characterize an overgrowth of Archaea throughout the intestinal tract (Pimentel M, Saad R J, Long M D, Rao S S C. Am J Gastroenterol. 2020; 115 (2):165-178; doi.org/10.14309/ajg.0000000000000501). IMO is an overgrowth of the Archaea group of microorganisms. Intestinal methanogen overgrowth confirmed by lactulose breath testing. The standard allopathic treatment for IMO is a regime of antibiotics to flush the system. The standard antibiotics used are rifaximin and either neomycin and/or metronidazole as the treatments of choice. Methanobrevibacter smithii is the predominant methanogen and evidence suggests a strong association between the presence of methane gas and constipation-predominant irritable bowel syndrome (IBS-C) (Kim G, Deepinder F, Morales W, et al., Dig Dis Sci. 2012; 57 (12): 3213-3218; doi: 10.1007/s10620-012-2197-1). The prevalence of SIBO is higher in people with IBS when compared to healthy controls (Chen B, Kim J J, Zhang Y, Du L, Dai N. J Gastroenterol. 2018; 53 (7): 807-818).

Methane productions in humans has been associated with obesity. In-depth examination of the GI microbiome has shown clear changes in the microbial community during obesity, and importantly, that these changes can drive metabolic changes (Turnbaugh P J et al (2009) Nature 457:480-484; Ridaura V K et al (2013) Science 341:1241214). Recently, the methanogenic archaea (methanogens) have been identified as a potential regulator of metabolic function. These microorganisms metabolize a variety of electron sources including dihydrogen gas and small fatty acids within the gut milieu to produce methane (reviewed in Pimentel M et al (2012) Am J Gastroenterol Suppl 1:28-33.). While not all humans have detectable levels of breath methane, studies have shown that methane production is altered in disease states. Indeed, It appears that faecal methanogen numbers are elevated during human obesity and that disrupting methanogens through antibiotic treatment of their bacterial syntrophs also corresponds to reduced insulin secretion (Mathur R et al (2016) Obesity 24:576-582). Other studies have demonstrated that intestinal methane production in obese individuals is associated with a higher body mass index, and that higher concentration of methane detected by breath testing is a predictor of significantly greater obesity in overweight subjects (Basseri R J et al (2012) Gastroenterology & Hepatology 8 (1): 22-28).

The compositions provided herein are applicable for use and in methods for alleviation of methane production in humans with intestinal disorders, obesity, high body mass index (BMI). In one embodiment, the intestinal disorders are selected from SIBO, IBO, IBS and IBS-C.

The compositions and methods described herein may be provided as feed additives. Probiotics advantageously provide a feed efficiency benefit.

Strains for use as probiotics or microbiome modulators in the compositions and methods herein include Bacillus and Lactobacillus spp., which are known to alter the microorganism make up or microbiome in an animal and also produce antimicrobial peptides (broad activities) and secondary metabolites which can lead to anti-methanogens activity. Anti-methanogen activities of Bacillus strains (B. amyloliquefaciens BE191006, BE202071, and B. subtilis BE191105) and Lactobacillus reuteri 3632) have been demonstrated. Without wishing to be bound by any particular theory, the mechanism by which the probiotics or microbiome modulators act varies depending on the probiotic strain.

The present disclosure provides compositions comprising Bacillus and Lactobacillus spp. for reducing methane emissions in a ruminant. Specifically, the present provides compositions comprising Bacillus strains such as B. amyloliquefaciens BE191006, BE202071, and B. subtilis BE191105, as well as compositions including Lactobacillus reuteri 3632, and combinations thereof, for reducing methane emissions in an animal. The present provides compositions comprising Bacillus strains such as B. amyloliquefaciens BE191006, BE202071, and B. subtilis BE191105, as well as compositions including Lactobacillus reuteri 3632, and combinations thereof, for reducing methane emissions in a ruminant animal. The present provides compositions comprising Bacillus strains such as B. amyloliquefaciens BE191006, BE202071, and B. subtilis BE191105, as well as compositions including Lactobacillus reuteri 3632, and combinations thereof, for reducing the production of methane in an animal. The present provides compositions comprising Bacillus strains such as B. amyloliquefaciens BE191006, BE202071, and B. subtilis BE191105, as well as compositions including Lactobacillus reuteri 3632, and combinations thereof, for reducing the production of methane in a ruminant animal. The present disclosure provides compositions comprising Bacillus strains such as B. amyloliquefaciens BE191006, BE202071, and B. subtilis BE191105, as well as compositions including Lactobacillus reuteri 3632, and combinations thereof, for reducing methane emissions in situations and locations where methanogenic Archaea or methanogenic bacteria are present or located.

Bacillus strains—exemplary and suitable Bacillus strains may be selected from one or more of Bacillus amyloliquefaciens strain and/or one or more Bacillus subtilis strain. In particular, exemplary Bacillus amyloliquefaciens strains are provided in Strain 06 (also denoted BE191006) and Strain 071 (also denoted BE202071). In particular, an exemplary Bacillus subtilis strain is provided in Strain 105 (also denoted BE191105). The strains are described below by reference in aspects. Details regarding these strains, including certain of their probiotic characteristics, their metabolites, including when cultured independently or in combinations, their unique genes, antibacterial peptides or enzymes, encoded proteins, and their genome nucleic acid sequence are provided and disclosed in PCT/US22/044211, based on U.S. Application Ser. No. 63/083,697 filed Sep. 25, 2020 and U.S. Application Ser. No. 63/241,369 filed Sep. 8, 2021, the disclosures of which are incorporated herein by reference in their entireties.

Strain 06: Bacillus amyloliquefaciens strain BE191006 (also denoted as ELA191006) corresponding to ATCC deposit PTA-127065. BE191006 corresponding to ATCC deposit PTA-127065 or a Bacillus strain having at least 90% identity, 95% identity, 97% identity, 98% identity, 99% identity in genomic sequence to the sequence of BE191006 corresponding to ATCC deposit PTA-127065. The genome nucleic acid sequence of strain BE191006 (Strain 06) is provided in SEQ ID NO: 27.

Strain 071: Bacillus amyloliquefaciens strain BE202071 (also denoted as ELA202071) corresponding to ATCC deposit PTA-127064. BE202071 corresponding to ATCC deposit PTA-127064 or a Bacillus strain having at least 90% identity, 95% identity, 97% identity, 98% identity, 99% identity in genomic sequence to the sequence of BE202071 corresponding to ATCC deposit PTA-127064. The genome nucleic acid sequence of strain BE202071 (strain 071) is provided in SEQ ID NO: 42.

Strain 105: Bacillus subtilis strain BE191105 (also denoted as ELA191105) corresponding to ATCC deposit PTA-126786. BE191105 corresponding to ATCC deposit PTA-126786 or a Bacillus strain having at least 90% identity, 95% identity, 97% identity, 98% identity, 99% identity in genomic sequence to the sequence of BE191105 corresponding to ATCC deposit PTA-126786. The genome nucleic acid sequence of strain BE191105 (strain 105) is provided in SEQ ID NOs: 1, 2, 3, 4 and/or 5.

In one embodiment, the isolated Bacillus amyloliquefaciens strain comprises strain BE191006 deposited with ATCC under patent deposit number PTA-127065. In one embodiment, the isolated Bacillus amyloliquefaciens strain comprises strain BE202071 deposited with ATCC under patent deposit number PTA-127064. In one embodiment, the isolated Bacillus subtilis strain comprises strain BE191105 deposited with ATCC under patent deposit number PTA-126786.

Bacillus amyloliquefaciens strain “BE191006” (denoted as ELA191006) was deposited on 11 May 2021 according to the Budapest Treaty in the American Type Culture Collection (ATCC), ATCC Patent Depository, 10801 University Boulevard, Manassas, Va., 20110, USA. The deposit has been assigned ATCC Patent Deposit Number PTA-127065.

Bacillus amyloliquefaciens strain “BE202071” (denoted as ELA202071) was deposited on 11 May 2021 according to the Budapest Treaty in the American Type Culture Collection (ATCC), ATCC Patent Depository, 10801 University Boulevard, Manassas, Va., 20110, USA. The deposit has been assigned ATCC Patent Deposit Number PTA-127064.

Bacillus subtilis strain “BE191105” (denoted as ELA191105) was deposited on 19 Jun. 2020 according to the Budapest Treaty in the American Type Culture Collection (ATCC), ATCC Patent Depository, 10801 University Boulevard, Manassas, Va., 20110, USA. The deposit has been assigned ATCC Patent Deposit Number PTA-126786.

Lactobacillus—exemplary and suitable Lactobacillus strains may be selected from one or more Lactobacillus reuteri strain. In particular, an exemplary Lactobacillus reuteri strain is provided in Strain 3632. The strain is described below by reference in aspects. Details regarding this strain, including certain of its probiotic characteristics, metabolites, unique genes, antibacterial peptides or enzymes, encoded proteins, and genome nucleic acid sequence are provided and disclosed in WO 2020/163398 published Aug. 13, 2020, which is incorporated herein by reference in their entireties.

Strain 3632: ATCC Patent Deposit Number PTA-126788. Isolated Lactobacillus strain PTA-126788 or a Lactobacillus strain having at least 99% amino acid or nucleic acid identity to strain PTA-126788. The genome nucleic acid sequence of strain 3632 and ATCC PTA 126788 is provided in SEQ ID NOs: 51-57.

In an embodiment, the composition, particularly a probiotic or microbiome modulator composition, comprises at least isolated Lactobacillus strain 3632 (PTA-126788) or a Lactobacillus strain having at least 99% amino acid or nucleic acid identity to strain 3632 (PTA-126788). In an embodiment, the composition, particularly a probiotic or microbiome modulator composition, comprises isolated Lactobacillus strain 3632 (PTA-126788) or a Lactobacillus strain having at least 99% amino acid or nucleic acid identity to strain 3632 (PTA-126788) and at least one Bacillus strain. In an embodiment, the composition, particularly a probiotic or microbiome modulator composition, comprises isolated Lactobacillus strain 3632 (PTA-126788) or a Lactobacillus strain having at least 99% amino acid or nucleic acid identity to strain 3632 (PTA-126788) and one Bacillus strain. In an embodiment, the composition, particularly a probiotic or microbiome modulator composition, comprises isolated Lactobacillus strain 3632 (PTA-126788) or a Lactobacillus strain having at least 99% amino acid or nucleic acid identity to strain 3632 (PTA-126788) and at least two Bacillus strains. In an embodiment, the composition, particularly a probiotic or microbiome modulator composition, comprises isolated Lactobacillus strain 3632 (PTA-126788) or a Lactobacillus strain having at least 99% amino acid or nucleic acid identity to strain 3632 (PTA-126788) and a Bacillus strain selected from strain 06 (PTA-127065), strain 071 (PTA-127064) and strain 105 (PTA-126786).

In an embodiment of the invention, a probiotic composition, microbiome modulator or direct feed microbial is provided which comprises at least one Bacillus strain. In an embodiment of the invention, a probiotic composition or direct feed microbial is provided which comprises a combination of at least two Bacillus strains. In embodiments, the Bacillus strain or strains are selected from Strain 06 (BE191006) or a Bacillus strain having at least 90% identity, 95% identity, 97% identity, 98% identity, 99% identity in genomic sequence to the sequence of Strain 06 (BE191006); Strain 71 (BE202071) or a Bacillus strain having at least 90% identity, 95% identity, 97% identity, 98% identity, 99% identity in genomic sequence to the sequence of Strain 71 (BE202071); and Strain 105 (BE191105) or a Bacillus strain having at least 90% identity, 95% identity, 97% identity, 98% identity, 99% identity in genomic sequence to the sequence of and Strain 105 (BE191105). In embodiments, the Bacillus strain or strains are selected from Strain 06 (BE191006) or a Bacillus strain having at least 90% identity, 95% identity, 97% identity, 98% identity, 99% identity in genomic sequence to the sequence of SEQ ID NO:27; Strain 71 (BE202071) or a Bacillus strain having at least 90% identity, 95% identity, 97% identity, 98% identity, 99% identity in genomic sequence to the sequence of SEQ ID NO:42; and Strain 105 (BE191105) or a Bacillus strain having at least 90% identity, 95% identity, 97% identity, 98% identity, 99% identity in genomic sequence to the sequence of one or more of SEQ ID NO: 1, 2, 3, 4 and/or 5.

In an embodiment of the invention, a probiotic composition, microbiome modulator or direct feed microbial is provided which comprises at least one Lactobacillus strain. In an embodiment of the invention, a probiotic composition or direct feed microbial is provided which comprises at least one Lactobacillus reuteri strain. In an embodiment of the invention, a probiotic composition or direct feed microbial is provided which comprises a combination of two Lactobacillus strains, particularly a combination of two Lactobacillus reuteri strains. In embodiments, the at least one Lactobacillus strain is selected from Strain 3632 (PTA-126788) or a Lactobacillus strain having at least 90% identity, 95% identity, 97% identity, 98% identity, 99% identity in genomic sequence to the sequence of Strain 3632 (PTA-126788); and Strain 3630 (PTA-126787). In embodiments, the at least one Lactobacillus strain is selected from Strain 3632 (PTA-126788) or a Lactobacillus strain having at least 90% identity, 95% identity, 97% identity, 98% identity, 99% identity in genomic sequence to the sequence of one or more of SEQ ID NO: 51, 52, 53, 54, 55, 56 and/or 57.

### Direct Feed Microbials (DFMs) as Delivery or Production Systems

Strains suitable as DFMs can provide an attractive and useful starting point for applications to produce or generate biomolecules and heterologous proteins, including as a live delivery system for synthesis and delivery of molecules or proteins with wide applications including in therapy and in animal health. These direct feed strains have applicability as a delivery system which can constantly deliver useful therapeutic molecules and biomolecules, such as anti-infective molecules, directly to the host, such as to the gastrointestinal tract, where bacteria, including pathogenic bacteria or methanogenic bacteria, are replicating in the host. The gastrointestinal system is also often a point of entry of the bacteria or pathogen into the host. Preferably, the delivery system is a live genetically-modified microorganism, such as a bacterium, which can reproduce in—and even colonize in some instances—a host and directly deliver therapeutic molecules and biomolecules, such as antiinfective, antipathogenic, antibacterial or anti-methanogen or methanogen reducing agents to reduce the number of, or block the entry of, a pathogen, a bacteria, or Archaea, or the amount of methane or such other molecule(s) produced. These bacterial strains provide improved delivery platforms and systems, including suitable vectors and nucleic acid-based systems for rapid and effective expression of heterologous proteins or genes of interest and robust generation of numerous vehicles using a single platform.

### Bacillus Subtilis—Strain 105

Bacillus subtilis is a Gram-positive model bacterium which is widely used for industrial production of recombinant proteins such as alpha-amylase, protease, lipase, and other industrial enzymes. Because of the ability of the bacteria to produce large amounts of a target protein, and also to secrete large amounts of a target protein into the culture medium, and the availability of a low-cost downstream production and purification process, over 60% of commercial industrial enzymes are produced in Bacillus subtilis and relative Bacillus species (Schallmey, M.; Singh, A.; Ward, O. P. (2004) 50 (1): 1-17). In contrast to the frequently used recombinant protein expression host Escherichia coli, Bacillus subtilis has no risk of endotoxin contamination and has been certificated as a GRAS (generally regarded as safe) organism by the FDA, which makes it a choice for food-grade and pharmaceutical protein production.

B. subtilis strains, particularly strain 105, provides a Bacillus subtilis expression system which can be modified and engineered to produce high levels of at least one or a multiplicity of biomolecules or heterologous proteins, including in instances as surface-displayed or secreted molecules. Bacillus subtilis strain BE191105, also denoted as ELA191105 and strain 105, corresponds to ATCC deposit PTA-126786. Strain 105 is described and detailed as a genetically modified strain for live delivery or production in PCT/US2022/044211 filed Sep. 21, 2022, which is based on U.S. Ser. No. 63/247,271 (filed Sep. 11, 2021), 63/247,273 (filed Sep. 22, 2021) and 63/247,400 (filed Sep. 23, 2021) (incorporated herein by reference). These applications describe native bacterial promoters, signal sequences suitable for expression and vectors and bacterial genome sites/genes for integration to generate stable modified strains, as well as modifications to strain 105 to improve expression.

B. subtilis strain 105 has been described as a microbial having beneficial effects, including in combination with one or more Bacillus amyloliquefaciens strain. In some aspects/embodiments, the B. subtilis strain 105 can be combined with one or more isolated Bacillus amyloliquefaciens strain, particularly selected from BE191024 (also denoted as ELA191024) (corresponding to ATCC deposit PTA-126784), BE191036 (also denoted as ELA191036) (corresponding to ATCC deposit PTA-126785), BE191006 (also denoted as ELA191006) (corresponding to ATCC deposit PTA-127065) and BE202071 (also denoted as ELA202071) (corresponding to ATCC deposit PTA-127064). These probiotic strain combinations and compositions and methods thereof are described and provided in PCT/US2021/051973 filed Sep. 24, 2021, published as WO2022/067052 Mar. 31, 2022 (incorporated herein by reference). Combinations of B. subtilis 105 and one or more B. amyloliquefaciens strain have been described for treating necrotic enteritis in poultry. The bacillus strains have efficacy in reducing mortality in poultry and for improving performance selected from average daily feed intake (ADFI), average daily gain (ADG) and feed conversion ratio (FCR) in poultry. The strain combinations are also effective in reducing post-weaning diarrhea in swine, and for improving feed intake, penn weight and/or weight gain in swine, as well as for improving performance selected from average daily feed intake (ADFI), average daily gain (ADG) and feed conversion ratio (FCR) in swine, particularly in post-weaning swine. No effects or activities regarding methanogenic Archaea and/or methanogenic bacteria or methanogenesis or methane production have been described.

Lactobacillus reuteri strains 3630 and 3632 are described and detailed as novel strains suitable as DFMs, including in combination, and also as suitable strains for genetic modification and as live delivery or production strains. Lactobacillus reuteri strain 3630 was deposited on 19 Jun. 2020 in the ATCC Patent Depository and assigned ATCC Patent Deposit Number PTA-126787. The L. reuteri strains 3630 and 3632 are described and detailed as probiotic strains in Probiotic Compositions Comprising Lactobacillus Reuteri Strains and Methods of Use PCT/US2020/016668 filed Feb. 4, 2020, published as WO 2020/163398 Aug. 13, 2020. Corresponding US publications are US 2022/0088094 published Mar. 24, 2022 and US 2022/0125860 published Apr. 28, 2022. A live delivery system based on L. reuteri strain 3630 or 3632 is described and detailed in A Genetically Modified Lactobacillus and Uses Thereof PCT/US2020/016522 filed Feb. 4, 2020, published as WO 2020/163284 Aug. 13, 2020. This application describes native bacterial promoters, signal sequences suitable for expression and vectors and bacterial genome sites/genes for integration to generate stable modified strains. These applications are incorporated herein by reference. No effects or activities regarding methanogenic Archaea and/or methanogenic bacteria or methanogenesis or methane production have been described.

In some aspects, the compositions described above are used to alter the microbiome or to reduce bacterial, microorganism or Archaea load, particularly pathogenic bacteria, methanogenic bacteria or organisms, or clinically significant bacteria, including the number or amount or type (genus, species) of bacteria or Archaea in the gut or gastrointestinal tract or rumen of an animal. The bacteria may particularly be a methanogenic bacteria or methanogenic Archaea selected, for example, from at least one of Methanobrevibacter ruminantium, Methanobacterium formicum, and Methanobacterium mobile.

In some aspects, the compositions described above are used to reduce transmission of bacteria or alter the flora or microbiome of microorganisms or bacteria, particularly pathogenic bacteria or methanogenic organisms or bacteria, in an animal pen or in a group or herd of animals. In some aspects, the compositions described above are used to reduce or alter transmission or alter the flora or microbiome of microorganisms or bacteria in an animal pen or in a group or herd of animals of at least one methanogenic bacteria or methanogenic Archaea, such as selected from, for example, Methanobrevibacter ruminantium, Methanobacterium formicum, and Methanobacterium mobile.

In embodiments of the invention, an animal may include a farmed animal or livestock or a domesticated animal. Livestock or farmed animal may include cattle (e.g., cows or bulls (including calves)), poultry (including broilers, chickens and turkeys), pigs (including piglets), birds, aquatic animals such as fish, agastric fish, gastric fish, freshwater fish such as salmon, cod, trout and carp, e.g. koi carp, marine fish such as sea bass, and crustaceans such as shrimps, mussels and scallops), horses (including race horses), sheep (including lambs). As used herein, the term “ruminants” includes, without limitation, extensive beef cattle, intensive beef cattle and dairy cattle. Ruminants also include sheep, buffalo, goats, bison. An animal may be a cat or dog. An animal may be a human.

The compositions may further include one or more component or additive. The one or more component or additive may be a component or additive to facilitate administration, for example by way of a stabilizer or vehicle, or by way of an additive to enable administration to an animal such as by any suitable administrative means, including in aerosol or spray form, in water, in feed or in an injectable form. Administration to an animal may be by any known or standard technique. These include oral ingestion, gastric intubation, or broncho-nasal spraying. The compositions disclosed herein may be administered by immersion, intranasal, intramammary, topical, mucosally, or inhalation.

In some embodiments, the composition does not include antibiotics. In some embodiments, the composition includes antibiotics. Exemplary antibiotics include tetracycline, bacitracin, tylosin, salinomycin, virginiamycin and bambermycin.

In some embodiments, the Bacillus strains of the present disclosure are not genetically engineered or genetically modified and do not contain heterologous genetic sequences. In some embodiments, the Lactobacillus strains of the present disclosure are not genetically engineered or genetically modified and do not contain heterologous genetic sequences.

The compositions described above may include a carrier suitable for animal consumption or use. Examples of suitable carriers include edible food grade material, mineral mixture, gelatin, cellulose, carbohydrate, starch, glycerin, water, glycol, molasses, corn oil, animal feed, such as cereals (barley, maize, oats, and the like), starches (tapioca and the like), oilseed cakes, and vegetable wastes. In some embodiments, the compositions include vitamins, minerals, trace elements, emulsifiers, aromatizing products, binders, colorants, odorants, thickening agents, and the like.

In some embodiments, the compositions include one or more biologically active molecule or therapeutic molecule. Examples of the aforementioned include ionophore; vaccine; antibiotic; antihelmintic; virucide; nematicide; amino acids such as methionine, glycine, and arginine; fish oil; krill oil; and enzymes.

In an embodiment, the composition includes one or more anti-methanogenic compound. In an embodiment, the composition is administered or fed in combination with one or more anti-methanogenic compound. In an embodiment, the composition includes one or more compound capable of reducing or inhibiting methane (CH4) production, release, or stability. In an embodiment, the composition is administered or fed in combination with one or more compound capable of reducing or inhibiting methane (CH4) production, release, or stability. Compositions of one or more strains provided herein with one or more anti-methanogenic compound selected from inhibitors of a rate-limiting enzyme in methanogenesis pathway, inhibitors of methanogens lipid biosynthesis, or microbiome modulators are contemplated and provided herein. Exemplary anti-methanogenic compounds or inhibitors of methane include inhibitors of enzyme methyl Coenzyme M reductase, such as include 3-NOP, bromoethanesulfonate, bromoform, red algae/Asparagopsis taxiformis, inhibitors of methanogens lipid biosynthesis, such as the inhibitor mevinolin, and/or microbiome modulators, such as monensin.

In some embodiments, the compositions or combinations may additionally include one or more prebiotic. In some embodiments, the compositions may be administered along with or may be coadministered with one or more prebiotic. Prebiotics may include organic acids or non-digestible feed ingredients that are fermented in the lower gut and may serve to select for beneficial bacteria. Prebiotics may include mannan-oligosaccharides, fructo-oligosaccharides, galacto-oligosaccharides, chito-oligosaccharides, isomalto-oligosaccharides, pectic-oligosaccharides, xylo-oligosaccharides, and lactose-oligosaccharides.

The compositions provided herein and products derived therefrom have application and use as postbiotics. Thus, the fermentation product of the strains and compositions described herein provides a postbiotic product and composition useful and with application in reducing methane production and greenhouse gases and altering methanogenesis. This can be applied in animals or outside of the body, such as in the environment (ponds, manure, soils, etc.) or a culture or process (anaerobic digester, waste treatment, etc.). Postbiotics refer to the waste or growth product remaining after prebiotics and probiotics are digested. Postbiotics can include nutrients such as vitamins, amino acids, antimicrobial peptides, fatty acids, such as short chain fatty acids or volatile fatty acids. Fermented foods are also exemplary postbiotics, such as kefir, tempeh and kimchi.

The probiotic or microbiome modulator compositions have application and uses outside of the body of an animal. These applications and uses include in the environment (ponds, manure, soils, etc.) or a culture or process (anaerobic digester, waste treatment, etc.). Administration or addition of a composition as provided herein can serve to alter methanogenesis and/or reduce methane production in the environment or in a culture or process. This can include GHG management environmentally, such as in manure or litter management. The compositions as prebiotic or the fermentation product of the compositions as postbiotics can be used in manure or litter management, including as additives thereto, to reduce methane gas production.

The compositions may be formulated as animal feed, feed additive, food ingredient, water additive, water-mixed additive, consumable solution, consumable spray additive, consumable solid, consumable gel, injection, or combinations thereof. The composition may be formulated and suitable for use as or in one or more of animal feed, feed additive, food ingredient, water additive, water-mixed additive, consumable solution, consumable spray additive, consumable solid, consumable gel, injection, or combinations thereof. The composition may be suitable and prepared for use as animal feed, feed additive, food ingredient, water additive, water-mixed additive, consumable solution, consumable spray additive, consumable solid, consumable gel, injection, or combinations thereof. When the compositions are administered as a feed additive, there is a feed efficiency benefit.

Compositions may include a carrier in which the bacterium or any such other components is suspended or dissolved. Such carrier(s) may be any solvent or solid or encapsulated in a material that is non-toxic to the inoculated animal and compatible with the organism. Suitable pharmaceutical carriers include liquid carriers, such as normal saline and other non-toxic salts at or near physiological concentrations, and solid carriers, such as talc or sucrose and which can also be incorporated into feed for farm animals. When used for administering via the bronchial tubes, the composition is preferably presented in the form of an aerosol. A dye may be added to the compositions hereof, including to facilitate checking or confirming whether an animal has ingested or breathed in the composition.

When administering to animals, including farm animals, administration may include orally or by injection. Oral administration can include by bolus, tablet or paste, or as a powder or solution in feed or drinking water. The method of administration will often depend on the species being feed or administered, the numbers of animals being fed or administered, and other factors such as the handling facilities available and the risk of stress for the animal.

In an embodiment, administered comprises in ovo administration. In an embodiment, administered comprises spray administration. In an embodiment, administered comprises immersion, intranasal, intramammary, topical, or inhalation.

The dosages required will vary and need be an amount sufficient to induce an immune response or to effect a biological or phenotypic change or response expected or desired. Routine experimentation will establish the required amount. Increasing amounts or multiple dosages may be implemented and used as needed.

In an embodiment of the invention, the bacterial strains are administered in doses indicated as CFU/g or colony forming units of bacteria per gram. In an embodiment, the dose is in the range of 1×10³ to 1×10⁹ CFU/g. In an embodiment, the dose is in the range of 1×10³ to 1×10⁷. In an embodiment, the dose is in the range of 1×10⁴ to 1×10⁶. In an embodiment, the dose is in the range of 5×10⁴ to 1×10⁶. In an embodiment, the dose is in the range of 5×10⁴ to 6×10⁵. In an embodiment, the dose is in the range of 7×10⁴ to 3×10⁵. In an embodiment, the dose is approximately 50K, 75K, 100K, 125K, 150K, 200K, 300K, 400K, 500K, 600K CFU/g.

In an embodiment, in instances where the composition, including a feed additive, comprises a combination of one or more strains of bacteria, the ratio of the first strain and the second or more strain arc in each instance a ratio of about 0.75-1.5:1. In an embodiment, in instances where the composition comprises a combination of one or more strains of bacteria, the ratio of the first strain and the second or more strain are about equal, about 1:1, about the same CFU dose of each strain. In an embodiment, in instances where the composition, including a feed additive, comprises a combination of one or more strains of bacteria, the amount of the CFU dose of the first strain and the second or more strain are each about equal. Thus, a composition comprising three strains, such as a combination of first isolated Bacillus amyloliquefaciens strain, second isolated Bacillus amyloliquefaciens strain, and isolated Bacillus subtilis strain, the combination comprises a ratio of the first isolated Bacillus amyloliquefaciens strain, the second isolated Bacillus amyloliquefaciens strain, and the isolated Bacillus subtilis strain of 0.75-1.5:1:0.75-1.5. In an embodiment, the composition comprises about equal amounts of the first isolated Bacillus amyloliquefaciens strain, the second isolated Bacillus amyloliquefaciens strain, and the isolated Bacillus subtilis strain. Thus, a composition comprising three strains, such as a combination of two isolated Bacillus strains and an isolated Lactobacillus strain, the combination comprises a ratio of the first isolated Bacillus strain, the second isolated Bacillus strain, and the isolated Lactobacillus subtilis strain of 0.75-1.5:1:0.75-1.5. In an embodiment, the composition comprises about equal amounts of the first and second isolated Bacillus strain and the isolated Lactobacillus subtilis strain. In an embodiment, and in instances where two Bacillus strains or a Bacillus strain and a Lactobacillus strain are included in a composition, the ratio of the first strain and the second or more strain are in each instance a ratio of about 0.75-1.5:1. In an embodiment, the ratio or amount is characterized by the number of viable spores per gram dry weight. In an embodiment, the composition comprises from about 104 to about 1010 viable spores per gram dry weight. In an embodiment, the composition comprises from about 106 to about 1010 viable spores per gram dry weight. In an embodiment, the composition comprises from about 106 to about 108 viable spores per gram dry weight.

In an embodiment, the isolated strains are not genetically engineered. In an embodiment, the isolated strains are genetically engineered.

In an embodiment, a method is provided for reducing or inhibiting or altering the colonization of an animal by a methanogenic bacterium or methanogenic Archaea, the method comprising administering to an animal an effective amount of a composition according to the invention. In an embodiment, a method is provided for reducing or inhibiting or altering the colonization of an animal by a methanogenic bacterium or methanogenic Archaea, the method comprising administering to an animal an effective amount of a composition comprising one or more of an isolated Bacillus amyloliquefaciens strain, an isolated Bacillus subtilis strain, or an isolated Lactobacillus strain, such as a Lactobacillus reuteri strain. In an embodiment, a method is provided for reducing or inhibiting or altering the colonization of an animal by a methanogenic bacterium or methanogenic Archaea, the method comprising administering to an animal an effective amount of a composition comprising one or more of isolated Bacillus amyloliquefaciens strain Strain 06, isolated Bacillus amyloliquefaciens strain Strain 071, and isolated Bacillus subtilis strain Strain 105. In an embodiment, a method is provided for reducing or inhibiting or altering the colonization of an animal by a methanogenic bacterium or methanogenic Archaea, the method comprising administering to an animal an effective amount of a composition comprising one or more of isolated Bacillus amyloliquefaciens strain Strain 06, isolated Bacillus amyloliquefaciens strain Strain 071, isolated Bacillus subtilis strain Strain 105, and isolated Lactobacillus reuteri strain 3632. In an embodiment, a method is provided for reducing or inhibiting or altering the colonization of an animal by a methanogenic bacterium or methanogenic Archaea, the method comprising administering to an animal an effective amount of a composition comprising two or more of isolated Bacillus amyloliquefaciens strain Strain 06, isolated Bacillus amyloliquefaciens strain Strain 071, isolated Bacillus subtilis strain Strain 105, and isolated Lactobacillus reuteri strain 3632. In an embodiment, a method is provided for reducing or inhibiting or altering the colonization of the rumen of a ruminant animal by a methanogenic bacterium or methanogenic Archaca, the method comprising administering to an animal an effective amount of a composition comprising two or more of isolated Bacillus amyloliquefaciens strain Strain 06, isolated Bacillus amyloliquefaciens strain Strain 071, isolated Bacillus subtilis strain Strain 105, and isolated Lactobacillus reuteri strain 3632.

In an embodiment, a method is provided for altering the colonization by methanogenic bacteria or methanogenic Archaea in a natural environment or anaerobic circumstance where methanogenic bacteria or methanogenic Archaea are present, the method comprising contacting or delivering an effective amount of a composition comprising one or more of isolated Bacillus amyloliquefaciens strain Strain 06, isolated Bacillus amyloliquefaciens strain Strain 071, isolated Bacillus subtilis strain Strain 105, and isolated Lactobacillus reuteri strain 3632. In an embodiment, a method is provided for altering the amount of methane produced by methanogenic bacteria or methanogenic Archaea in a natural environment or anaerobic circumstance where methanogenic bacteria or methanogenic Archaea are present, the method comprising contacting or delivering an effective amount of a composition comprising one or more of isolated Bacillus amyloliquefaciens strain Strain 06, isolated Bacillus amyloliquefaciens strain Strain 071, isolated Bacillus subtilis strain Strain 105, and isolated Lactobacillus reuteri strain 3632.

In an embodiment, a method is provided for reducing or inhibiting or altering the colonization of an animal by a methanogenic bacterium or methanogenic Archaea, the method comprising administering to an animal an effective amount of a composition comprising an isolated Lactobacillus strain. In an embodiment, a method is provided for reducing or inhibiting or altering the colonization of an animal by a methanogenic bacterium or methanogenic Archaea, the method comprising administering to an animal an effective amount of a composition comprising an isolated Lactobacillus reuteri strain. In an embodiment, a method is provided for reducing or inhibiting or altering the colonization of an animal by a methanogenic bacterium or methanogenic Archaea, the method comprising administering to an animal an effective amount of a composition comprising an isolated Lactobacillus reuteri strain. In an embodiment, a method is provided for reducing or inhibiting or altering the colonization in the rumen of a ruminant animal by a methanogenic bacterium or methanogenic Archaea, the method comprising administering to a ruminant animal an effective amount of a composition comprising an isolated Lactobacillus strain. In an embodiment, a method is provided for reducing or inhibiting or altering the colonization in the rumen of a ruminant animal by a methanogenic bacterium or methanogenic Archaea, the method comprising administering to a ruminant animal an effective amount of a composition comprising an isolated Lactobacillus reuteri strain. In an embodiment, the method comprises administering to an animal an effective amount of Strain 3632. In an embodiment, the method comprises administering to an animal an effective amount of Strain 3632 and one or more Bacillus strain. In an embodiment, the method comprises administering to an animal an effective amount of Strain 3632 and one or more Bacillus strain selected from strain 06, strain 071 and strain 105.

In some embodiments, the compositions described above are used to reduce bacterial or microorganism load, particularly methanogenic bacteria or Archaea or clinically significant bacteria, including the number or amount of bacteria in the gut or gastrointestinal tract or rumen of an animal. In some embodiments, the compositions described above are used to alter bacterial or microorganism load or to alter the bacterial or microorganism composition or microbiome components, particularly methanogenic bacteria and/or Archaea or environmentally significant bacteria, including the number or amount or types or species or relative amounts and makeup of bacteria and/or Archaeain the gut or gastrointestinal tract or rumen of an animal.

In an embodiment, the method results in disrupting or altering the methane output or methane production or amount of methane produced by methanogenic bacteria and/or methanogenic Archaea. In an embodiment, the method results in inhibiting growth or killing the methanogenic bacteria and/or methanogenic Archaea. In an embodiment, the method results altering the fermentation processes of the methanogenic bacteria and/or methanogenic Archaea, such that the amount of methane produced or released is controlled or reduced. In an embodiment, the method results in reducing the methane output or methane production or amount of methane produced by an animal. In an embodiment, the method results in inhibiting growth or killing the methanogenic bacteria and/or methanogenic Archaea in an animal. In an embodiment, the method results altering the fermentation processes of the methanogenic bacteria and/or methanogenic Archaea, such that the amount of methane produced or released by an animal is controlled or reduced. In an embodiment, the method results in reducing the methane output or methane production or amount of methane produced by a ruminant animal. In an embodiment, the method results in inhibiting growth or killing the methanogenic bacteria and/or methanogenic Archaea in a ruminant animal. In an embodiment, the method results altering the fermentation processes of the methanogenic bacteria and/or methanogenic Archaea, such that the amount of methane produced or released by a ruminant animal is controlled or reduced.

In an embodiment of the method(s), the composition is formulated as animal feed, feed additive, food ingredient, water additive, water-mixed additive, consumable solution, consumable spray additive, consumable solid, consumable gel, injection, or combinations thereof. In an embodiment, the composition comprises animal feed.

The compositions described above may include a carrier suitable for animal consumption or use. Examples of suitable carriers include edible food grade material, mineral mixture, gelatin, cellulose, carbohydrate, starch, glycerin, water, glycol, molasses, corn oil, animal feed, such as cereals (barley, maize, oats, and the like), starches (tapioca and the like), oilseed cakes, and vegetable wastes. In some embodiments, the compositions include vitamins, minerals, trace elements, emulsifiers, aromatizing products, binders, colorants, odorants, thickening agents, and the like.

In some embodiments, the compositions include one or more biologically active molecule or therapeutic molecule. Examples of the aforementioned include ionophore; vaccine; antibiotic; antihelmintic; virucide; nematicide; amino acids such as methionine, glycine, and arginine; fish oil; krill oil; and enzymes.

In some embodiments, the compositions or combinations may additionally include one or more prebiotic. In some embodiments, the compositions may be administered along with or may be coadministered with one or more prebiotic. Prebiotics may include organic acids or non-digestible feed ingredients that are fermented in the lower gut and may serve to select for beneficial bacteria. Prebiotics may include mannan-oligosaccharides, fructo-oligosaccharides, galacto-oligosaccharides, chito-oligosaccharides, isomalto-oligosaccharides, pectic-oligosaccharides, xylo-oligosaccharides, and lactose-oligosaccharides.

The composition may be formulated as animal feed, feed additive, food ingredient, water additive, water-mixed additive, consumable solution, consumable spray additive, consumable solid, consumable gel, injection, or combinations thereof. The composition may be formulated and suitable for use as or in one or more of animal feed, feed additive, food ingredient, water additive, water-mixed additive, consumable solution, consumable spray additive, consumable solid, consumable gel, injection, or combinations thereof. The composition may be suitable and prepared for use as animal feed, feed additive, food ingredient, water additive, water-mixed additive, consumable solution, consumable spray additive, consumable solid, consumable gel, injection, or combinations thereof.

In embodiments of the invention, an animal may include a farmed animal or livestock or a domesticated animal. Livestock or farmed animal may include cattle (e.g. cows or bulls (including calves)), poultry (including broilers, chickens and turkeys), pigs (including piglets), birds, aquatic animals such as fish, agastric fish, gastric fish, freshwater fish such as salmon, cod, trout and carp, e.g. koi carp, marine fish such as sea bass, and crustaceans such as shrimps, mussels and scallops), horses (including race horses), sheep (including lambs). A domesticated animal may be a pet or an animal maintained in a zoological environment and may include any relevant animal including canines (e.g. dogs), felines (e.g. cats), rodents (e.g. guinea pigs, rats, mice), birds, fish (including freshwater fish and marine fish), and horses. In embodiments, the animal is a ruminant animal. In embodiments, the ruminant is a bovine. In embodiments, the ruminant animal is a cattle or a bison. In embodiments, the ruminant is a bovine, goat, sheep, giraffe, gazelle or antelope. In embodiments, the ruminant is a bovine, goat or sheep. In embodiments, the animal is a non-ruminant animal.

The animal may be a pregnant or breeding animal. The animal may be a pregnant ruminant animal. The animal may be a pregnant cow or cattle, a pregnant sow or a pregnant pig.

As used herein, “isolated” means that the subject isolate has been separated from at least one of the materials with which it is associated in a particular environment, for example, its natural environment.

Thus, an “isolate” does not exist in its naturally occurring environment; rather, it is through the various techniques known in the art that the microbe has been removed from its natural setting and placed into a non-naturally occurring state of existence. Thus, the isolated strain or isolated microbe may exist as, for example, a biologically pure culture in association with an acceptable carrier.

As used herein, “individual isolates” should be taken to mean a composition, or culture, comprising a predominance of a single species, or strain, of microorganism, following separation from one or more other microorganisms. The phrase should not be taken to indicate the extent to which the microorganism has been isolated or purified. However, “individual isolates” can include substantially only one species, or strain, of microorganism.

In certain aspects of the disclosure, the isolated Bacillus strain and/or the isolated Lactobacillus strain exists as isolated and biologically pure cultures. It will be appreciated by one of skill in the art, that an isolated and biologically pure culture of a particular Bacillus strain or Lactobacillus strain, denotes that said culture is substantially free (within scientific reason) of other living organisms and contains only the individual bacillus and/or lactobacillus strain in question. The culture can contain varying concentrations of said isolated bacillus and/or lactobacillus strain. The present disclosure notes that isolated and biologically pure microbes often necessarily differ from less pure or impure materials.

In some embodiments of the present invention, the composition includes a combination of two isolated bacterial strains. In some embodiments of the present invention, the composition includes a combination of two isolated Bacillus strains. In some embodiments of the present invention, the composition includes a combination of two or more isolated bacterial strains. In some embodiments of the present invention, the composition includes a combination of two or more isolated Bacillus or Lactobacillus strains. In some embodiments of the present invention, the composition includes a combination of three isolated Bacillus strains. In some embodiments of the present invention, the composition includes a combination of an isolated Bacillus strain and a Lactobacillus strain. In some embodiments of the present invention, the composition includes a combination of at least one isolated Bacillus strain and a Lactobacillus strain.

As used herein, the term “bacterial consortia”, “bacterial consortium”, “microbial consortia” or “microbial consortium” refers to a subset of a microbial community of individual microbial species, or strains of a species, which can be described as carrying out a common function, or can be described as participating in, or leading to, or correlating with, a recognizable parameter, such as a phenotypic trait of interest (e.g. increased feed efficiency in poultry). The community may comprise two or more species, or strains of a species, of microbes. In some instances, the microbes coexist within the community symbiotically.

As used herein, “spore” or “spores” refer to structures produced by bacteria that are adapted for survival and dispersal. Spores are generally characterized as dormant structures; however, spores are capable of differentiation through the process of germination. Germination is the differentiation of spores into vegetative cells that are capable of metabolic activity, growth, and reproduction. The germination of a single spore results in a single bacterial vegetative cell. Bacterial spores are structures for surviving conditions that may ordinarily be nonconductive to the survival or growth of vegetative cells.

The composition may include or comprise live bacteria or bacterial spores, or a combination thereof.

As used herein, the terms “colonize” and “colonization” include “temporarily colonize” and “temporary colonization”.

As used herein, “microbiome” refers to the collection of microorganisms that inhabit the gastrointestinal tract of an animal and the microorganisms' physical environment (i.e., the microbiome has a biotic and physical component). The microbiome is fluid and may be modulated by numerous naturally occurring and artificial conditions (e.g., change in diet, disease, antimicrobial agents, influx of additional microorganisms, etc.). The modulation of the gastrointestinal microbiome can be achieved via administration of the compositions of the disclosure can take the form of: (a) increasing or decreasing a particular Family, Genus, Species, or functional grouping of a microbe (i.e., alteration of the biotic component of the gastrointestinal microbiome) and/or (b) increasing or decreasing gastrointestinal pH, increasing or decreasing volatile fatty acids in the gastrointestinal tract, increasing or decreasing any other physical parameter important for gastrointestinal health (i.e., alteration of the abiotic component of the gut microbiome).

As used herein, “probiotic” or “microbe modulator” refers to a substantially pure microbe (i.e., a single isolate) or a mixture of desired microbes, and may also include any additional components (e.g., carrier) that can be administered to an animal or environment to provide a beneficial health effect or to alter the microbiome of/in an animal or in/or an environment. Probiotics or microbial or microbiome modulator compositions of the invention may be administered with an agent or carrier to allow the microbes to survive the environment, such as that of the gastrointestinal tract, i.e., to resist low pH and to grow in the gastrointestinal environment.

The term “growth medium” as used herein, is any medium which is suitable to support growth of a microbe. By way of example, the media may be natural or artificial including gastrin supplemental agar, minimal media, rich media, LB media, blood serum, and tissue culture gels. It should be appreciated that the media may be used alone or in combination with one or more other media. It may also be used with or without the addition of exogenous nutrients.

As used herein, “improved” should be taken broadly to encompass improvement of a characteristic of interest, as compared to a control group, or as compared to a known average quantity associated with the characteristic in question. For example, “improved” feed efficiency associated with application of a beneficial microbe, or microbial ensemble, of the disclosure can be demonstrated by comparing the feed efficiency of poultry treated by the microbes taught herein to the feed efficiency of poultry not treated. In the present disclosure, “improved” does not necessarily demand that the data be statistically significant (i.e. p<0.05); rather, any quantifiable difference demonstrating that one value (e.g. the average treatment value) is different from another (e.g. the average control value) can rise to the level of “improved.”

As used herein, the term “metabolite” refers to an intermediate or product of metabolism. In some embodiments, a metabolite includes a small molecule. Metabolites have various functions, including in fuel, structural, signaling, stimulatory and inhibitory effects on enzymes, as a cofactor to an enzyme, in defense, and in interactions with other organisms (such as pigments, odorants and pheromones). A primary metabolite is directly involved in normal 5 growth, development and reproduction. A secondary metabolite is not directly involved in these processes but usually has an important ecological function. Examples of metabolites include but are not limited to antibiotics and pigments such as resins and terpenes, etc. Metabolites, as used herein, include small, hydrophilic carbohydrates; large, hydrophobic lipids and complex natural compounds.

As used herein, “carrier”, “acceptable carrier”, or “pharmaceutical carrier” are used interchangeably and refer to a diluent, adjuvant, excipient, or vehicle with which the compound is administered. Such carriers can be sterile liquids, such as water and oils, including those of petroleum, animal, vegetable, or synthetic origin; such as peanut oil, soybean oil, mineral oil, sesame oil, and the like. Water or aqueous solution saline solutions and aqueous dextrose and glycerol solutions are preferably employed as carriers, in some embodiments as injectable solutions. Alternatively, the carrier can be a solid dosage form carrier, including but not limited to one or more of a binder (for compressed pills), a glidant, an encapsulating agent, a flavorant, and a colorant. The choice of carrier can be selected with regard to the intended route of administration and standard pharmaceutical practice. See Handbook of Pharmaceutical Excipients, (Sheskey, Cook, and Cable) 2017, 8th edition, Pharmaceutical Press; Remington's Pharmaceutical Sciences, (Remington and Gennaro) 1990, 18th edition, Mack Publishing Company; Development and Formulation of Veterinary Dosage Forms (Hardee and Baggot), 1998, 2nd edition, CRC Press.

As used herein, “delivery” or “administration” means the act of providing a beneficial activity to a host. The delivery may be direct or indirect. An administration could be by an oral, nasal, or mucosal route. For example without limitation, an oral route may be an administration through drinking water, a nasal route of administration may be through a spray or vapor, and a mucosal route of administration may be through direct contact with mucosal tissue. Mucosal tissue is a membrane rich in mucous glands such as those that line the inside surface of the nose, mouth, esophagus, trachea, lungs, stomach, gut, intestines, and anus. In the case of birds, administration may be in ovo, i.e. administration to a fertilized egg. In ovo administration can be via a liquid which is sprayed onto the egg shell surface, or an injected through the shell.

As used herein, the terms “treating”, “to treat”, or “treatment”, include restraining, slowing, stopping, inhibiting, reducing, ameliorating, or reversing the progression or severity of an existing symptom, disorder, condition, or disease. A treatment may also be applied prophylactically to prevent or reduce the incidence, occurrence, risk, or severity of a clinical symptom, disorder, condition, or disease.

As used herein, “animal” includes bird, poultry, a human, or a non-human mammal. Specific examples include chickens, turkey, dogs, cats, cattle, buffalo, bison, sheep, goats, deer, giraffes, gazelles, antelopes, salmon, fish, swine and horse. Particular examples include livestock animals, such as chickens, turkey, cattle, swine, sheep. The chicken may be a broiler chicken, egg-laying, or egg-producing chicken. As used herein, the term “poultry” includes domestic fowl, such as chickens, turkeys, ducks, and geese. The term “animal” includes a ruminant animal or a non-ruminant animal.

As used herein, “gut” refers to the gastrointestinal tract including stomach, small intestine, and large intestine. The term “gut” may be used interchangeably with “gastrointestinal tract”.

As used herein, a “genetically-modified microorganism” means any microorganism which has been altered from the natural state using molecular biological techniques. A genetic modification could be the deletion of a portion of the bacterial chromosome or a naturally-occurring plasmid. The genetic modification could also be the introduction of an artificial or exogenous nucleic acid into a portion of the chromosome. The introduction may or may not disturb or perturb the expression of a bacterial gene. The genetic modification could also be the introduction of an artificial plasmid. The genetically-modified microorganism may be a bacterium, a virus, a yeast, a mold, or a single-celled organism.

An “artificial nucleic acid” or “artificial plasmid” is any nucleic acid or plasmid which does not occur naturally, but rather has been constructed using molecular biological techniques. Portions of the nucleic acid or plasmid may occur naturally, but the those portions are in an artificial rBEtionship or organization.

As used herein, an “expression cassette” is an artificial nucleic acid constructed to result in the expression of a desired biomolecule by the genetically-modified microorganism. An expression cassette comprises one or more of a promoter for transcriptional expression, a nucleic acid sequence encoding a signal sequence for secretion, a nucleic acid sequence encoding a cell-wall anchor, at least one heterologous coding region encoding a desired biomolecule, a nucleic acid sequence encoding an expressed peptide tag for detection, and terminators for translation and transcription termination. A promoter directs the initiation of transcription of the coding regions into a messenger RNA and the translation of the mRNA into a peptide. A signal sequence for secretion, or a secretion signal sequence, directs the peptide to be located outside the cell membrane. The extracellular peptide could be a soluble, secreted protein or it may be cell-associated, particularly if the expression cassette contains a cell wall anchor sequence which attaches the extracellular peptide to a bacterial cell wall. An expressed peptide tag is any amino acid sequence which may be recognized by an antibody or other binding protein. The expressed peptide tag may also bind an inorganic substance, such as a six-histidine tag which binds to nickel molecules. Terminators for translation may be a stop codon or a spacer open reading frame containing a stop codon.

As used herein, a “heterologous coding region” is a nucleic acid sequence containing an open reading frame which encodes a peptide. The coding region is heterologous to the associated promoter, meaning the coding region and the promoter are not associated in their natural states.

A “heterologous” region of a nucleic acid, RNA or DNA, construct is an identifiable segment of RNA or DNA within a larger RNA or DNA molecule that is not found in association with the larger molecule in nature. Thus, when the heterologous region encodes a gene, the gene will usually be flanked by RNA or DNA that does not flank the genomic RNA or DNA in the genome of the source organism.

As used herein, a “protein” is a sequence of amino acids which assumes a three-dimensional structure. A “peptide” can be used interchangeably with protein, but may also be a short linear sequence of amino acids without a defined three-dimensional structure.

As used herein, a “desired biomolecule” is any molecule or peptide which may be advantageous to a host when administered via a live delivery platform. The desired biomolecule may be a peptide with anti-infective activity, a probiotic factor, an immunomodulatory factor, an anti-antinutritional factor, or a growth-promoting biomolecule. The desired biomolecule may also be an enzyme which produces a substance with anti-infective activity or a probiotic factor such as a vitamin.

As used herein, “anti-infective activity” includes any activity which prevents infection of a host with a pathogenic organism. The following molecules are examples of biomolecules possessing anti-infective activity: an antibacterial peptide; a lysin or lytic enzyme; a prophage, phage or virus; an enzyme, for example one that cleaves or disables a protein made by a pathogen; and an antibody which blocks, inhibits, or clears a pathogenic molecule. An anti-infective may have bactiostatic activity, which slows, reduces, or prevents the growth of a pathogenic species. A non-limiting example of an antibacterial peptide is a member of the mersacidin family or a mersacidin-like molecule, such as those described in EP0700998. A non-limiting example of lysins are lytic molecules produced by phage. Lysins may have specificity for certain pathogenic species of bacteria and have been suggested for use in substitution for traditional antibiotics. V. A. Fischetti, Viruses, vol. 10, no. 310 (2018); and R. Vazquez et al. Frontiers in Immunology, vol. 9, article 2252 (2018).

As used herein, a “probiotic factor” is a substance which, when produced by a microorganism, proves beneficial to a host. The probiotic factor may be an attachment molecule or an agglutinizing molecule which promotes colonization of the host with the microorganism and/or prolongs the period of time where the microorganism colonizes the host. The longer the microorganism persists in the host the longer the beneficial effect is provided.

As used herein, an “immunomodulatory factor” could be a cytokine, lymphokine, chemokine, interleukin, interferon, a colony stimulating factor, or a growth factor. The immunomodulatory factor could provide nonspecific enhancement of an immune response or the immunomodulatory factor could increase the number or tissue distribution of immune cells present in the host. The immunomodulatory factor may also reduce an inappropriate immune response, such as without limitation an autoimmune response.

As used herein, a “growth-promoting biomolecule” could be a growth factor, a transfer factor (such as an iron-chelating molecule), a hormone, or any other factor which promotes healthy metabolic activity.

As used herein, an “anti-nutritional factor” could include protease inhibitors for example, a trypsin inhibitors.

As used herein, “subject” includes bird, poultry, fish, a human, or a non-human animal. Specific examples include chickens, turkey, dogs, cats, cattle, buffalo, bison, sheep, goats, deer, giraffes, gazelles, antelopes, salmon, fish, swine and horse. Particular examples include livestock animals, such as chickens, turkey, cattle, swine, sheep. The chicken may be a broiler chicken, egg-laying, or egg-producing chicken. As used herein, the term “poultry” includes domestic fowl, such as chickens, turkeys, ducks, and geese. The term “subject” includes a ruminant animal or a non-ruminant animal.

The term “primer” as used herein refers to an oligonucleotide, whether occurring naturally as in a purified restriction digest or produced synthetically, which is capable of acting as a point of initiation of synthesis when placed under conditions in which synthesis of a primer extension product, which is complementary to a nucleic acid strand, is induced, i.e., in the presence of nucleotides and an inducing agent such as a DNA polymerase and at a suitable temperature and pH. The primer may be either single-stranded or double-stranded and must be sufficiently long to prime the synthesis of the desired extension product in the presence of the inducing agent. The exact length of the primer will depend upon many factors, including temperature, source of primer and use of the method. For example, for diagnostic applications, depending on the complexity of the target sequence, the oligonucleotide primer typically contains 15-25 or more nucleotides, although it may contain fewer nucleotides.

The primers herein are selected to be “substantially” complementary to different strands of a particular target DNA sequence. This means that the primers must be sufficiently complementary to hybridize with their respective strands. Therefore, the primer sequence need not reflect the exact sequence of the template. For example, a non-complementary nucleotide fragment may be attached to the 5′ end of the primer, with the remainder of the primer sequence being complementary to the strand. Alternatively, non-complementary bases or longer sequences can be interspersed into the primer, provided that the primer sequence has sufficient complementarity with the sequence of the strand to hybridize therewith and thereby form the template for the synthesis of the extension product.

As used herein, the term “mutant”, refers to a variation in a nucleic acid or DNA or RNA sequence or in a chromosome structure from that which is considered a normal or wild-type sequence or chromosome without defect. In the context of a nucleic acid, DNA or RNA sequence, examples of mutations include point mutations, insertions, and deletions. A deletion includes deletion of a part or entire gene. Such mutations may have functional effects such as, for example, a decrease in function of a gene product, ablation of function in a gene product, and/or a new or altered function in a gene product.

As used herein, “mutation” includes any alteration in one or more nucleic acids in a genomic sequence, including one or more base changes, deletions, and/or insertions, that result in silent mutations, non-sense mutations, mis-sense mutations, or any such other mutations that result in reduced function of a gene or result in an inactive or otherwise non-functional protein encoded by a gene. Mutations include but are not limited to mutations that result in premature stop codons, aberrant splicing, altered or failed transcription, or altered or failed translation. A gene comprising a mutation can have more than one mutation. Mutations include deletion of a gene or a significant portion of a gene, particularly such that the gene's protein is not produced or expressed and/or is inactive. Mutations include insertions, such as wherein a foreign or heterologous sequence or nucleic acid is introduced into or otherwise inserted in the gene. Such insertion may block or eliminate translation to active or full length protein, or may result in a significantly altered and distinct protein that is not active as the wild type. An insertion may facilitate isolation, detection, selection of the gene mutant, such as by introduction or insertion of an antibiotic resistance gene or a detectable marker or protein. In particular embodiments of the invention and as described herein, the mutation, including one or more mutation, is a non-natural mutation and is genetically engineered or recombinantly generated. In some embodiments, the mutation is genetically engineered or generated recombinantly in vitro. In some embodiments, the mutation is genetically engineered or generated recombinantly in a cell.

In some embodiments, a mutation is generated whereby a gene, or a large or significant portion of a gene or protein encoding nucleic acid, is deleted. In embodiments, one or more gene or a large or significant portion of a gene or protein encoding nucleic acid is deleted for example via recombination methods. Recombination methods for targeted deletion of genes are known and available to one skilled in the art. Such methods include homologous recombination, such as via an introduced plasmid, phage, or nucleic acid such as DNA or linear DNA fragment(s), recombination enzymes or recombinase enzyme mediated recombination, for example via recombinase recognition or target sequences, transposon mediated recombination and gene replacement.

There are various peptides or proteins which act independently as therapeutic biomolecules. Among these are anti-infective or anti-bacterial peptides which can serve to block or treat infection by infectious agents or bacteria.

In some embodiments, the disclosure provides for the use of any of the compositions described above in a therapy or treatment or to improve a phenotypic trait in an animal. In embodiments of the invention, an animal may be a ruminant or non-ruminant animal. In embodiments of the invention, an animal may include a farmed animal or livestock or a domesticated animal. The animal may be a pregnant or breeding animal, such as a pregnant sow or a pregnant pig.

Examples of improving a phenotypic trait includes improving growth or growth characteristics, improving feed efficiency, reducing methanogenesis or diverting the fermentation pathway away from methane production, increasing H₂ consumption, reducing H₂ production, increasing volatile fatty acid production, and increasing gut health or characteristic (reducing permeability and inflammation). In particular, examples include reducing methanogenesis or diverting the fermentation pathway away from methane production, increasing H₂ consumption, reducing H₂ production, increasing volatile fatty acid production, and increasing gut health or characteristic (reducing permeability and inflammation). In particular, examples include one or more of reducing methanogenesis or diverting the fermentation pathway away from methane production, increasing H₂ consumption, reducing H₂ production, increasing volatile fatty acid production, and increasing gut health or characteristic (reducing permeability and inflammation).

The compositions may further include one or more component or additive. The one or more component or additive may be a component or additive to facilitate administration, for example by way of a stabilizer or vehicle, or by way of an additive to enable administration to an animal such as by any suitable administrative means, including in aerosol or spray form, in water, in feed or in an injectable form. Administration to an animal may be by any known or standard technique. These include oral ingestion, gastric intubation, or broncho-nasal spraying. The compositions disclosed herein may be administered by immersion, intranasal, intramammary, topical, mucosally, or inhalation. When the animal is a bird the treatment may be administered in ovo or by spray inhalation.

Any examples or illustrations given herein are not to be regarded in any way as restrictions on, limits to, or express definitions of any term or terms with which they are utilized. Instead, these examples or illustrations are to be regarded as being described with respect to one particular embodiment and as being illustrative only. Those of ordinary skill in the art will appreciate that any term or terms with which these examples or illustrations are utilized will encompass other embodiments which may or may not be given therewith or elsewhere in the specification and all such embodiments are intended to be included within the scope of that term or terms. Language designating such nonlimiting examples and illustrations includes, but is not limited to: “for example,” “for instance,” “e.g.,” and “in one embodiment.” In this specification, groups of various parameters containing multiple members are described. Within a group of parameters, each member may be combined with any one or more of the other members to make additional sub-groups. For example, if the members of a group are a, b, c, d, and c, additional sub-groups specifically contemplated include any one, two, three, or four of the members, e.g., a and c; a, d, and e; b, c, d, and e; etc.

Throughout this specification, quantities are defined by ranges, and by lower and upper boundaries of ranges. Each lower boundary can be combined with each upper boundary to define a range. The lower and upper boundaries should each be taken as a separate element. Two lower boundaries or two upper boundaries may be combined to define a range.

The present disclosure may be better understood with reference to the examples, set forth below. The following examples are put forth so as to provide those of ordinary skill in the art with a complete disclosure and description of how the compounds, compositions, and/or methods claimed herein are made and evaluated, and are intended to be purely exemplary and are not intended to limit the disclosure. It will be appreciated that other embodiments and uses will be apparent to those skilled in the art and that the invention is not limited to these specific illustrative examples or preferred embodiments.

### Example 1

### Mitigation of Enteric Methane Emission for Improved Livestock Production

Methane is naturally produced as a by-product of the ruminal microbial fermentation process (removal H₂, a thermodynamic block for fermentation) by methanogens. Methane emission is considered loss of energy for animals. Thus, reduction of methane should translate to improved feed efficiency. We have undertaken approaches for reduction of enteric methane gas emission to reduce the carbon footprint of livestock production and improve ruminant feed efficiency. Methane production by volume of biogas is evaluated and specific methanogenic activity estimated from methane production.

In an in vitro rumen batch culture, which provides an ‘ex vivo’ rumen evaluation system, several Bacillus and Lactobacillus strains reduced methane. In particular B. subtilis strain BE191006 (BE191006) demonstrated up to a 25% reduction in methane compared to control. Other strains reduced methane by at least 10%.

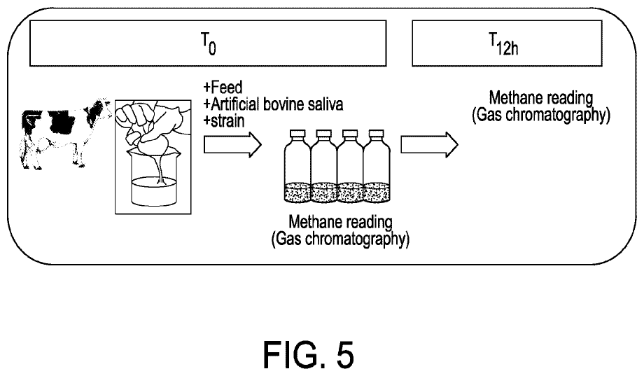

The experimental design of the rumen batch culture is depicted in FIG. 5. Rumen probiotic experiments evaluating the capability and activity of bacteria strains to reduce methane production in an in vitro rumen batch culture, which provides an ‘ex vivo’ rumen evaluation system, are conducted as follows. This system can be utilized to evaluate single strains or combinations of strains, one or more strain in combination with another agent, etc.

### Step 1: Feed Preparation

1. Beef cattle feed was dried in an oven at 58° C. for 96 h.
2. The dried feed was grinded using Oster 10-Cup Food Processor (precleaned with 70% IPA) at high speed for 5 min and the grinded feed was filtered through a Cooking Concept® mesh strainer (precleaned with 70% IPA) to separate the fine and grainy feed.
3. The fine feed was used immediately or stored at −20° C.

### Step 2: Serum Bottle Preparation

1. Serum bottle was cleaned using a MayTag dishwasher with jet clean cycle and tough scrub, hi temp wash, heated dry, extra dry, and steam sanitize cleaning addition.
2. The cleaned serum bottle was autoclaved for 1 h and stored at RT.
3. Two day before the experiment, 10% (w/v) fine feed was added to the precleaned serum bottle.

### Step 3: Probiotics and Additives Preparation

1. One day before the experiment, all powdered formed probiotic were added to the prefilled serum bottles
CaCO₃, Bacillus strains strain BE191105, strain BE191006, and strain BE202071
2. On the day of the experiment, all liquid formed probiotic were added to the prefilled serum bottles.
3. CHCl₃, Lactobacillus strain BE3632 (L. reuteri) and another strain denoted Strain A
4. On the day of the experiment, serum bottles that contained fine feed (10% w/v) and probiotic (1×10⁸ CFU per g feed) or enzymes were filled with 20 mL artificial saliva, stoppered with rubber stopper, and made anaerobic with N₂:CO₂ (80:20, v/v) gas for 3 cycles (with vacuum time of 3-3-5 minutes).
5. The ready mixture was filled with N₂:CO₂ (80:20, v/v) gas to 5 psig.

### Step 4: Rumen Fluid Preparation

1. Rumen contents were collected from fistulated cows in a centrifuged bottle with a sealing cap.
2. The centrifuged bottles with the rumen contents were transferred inside an anaerobic chamber filled with N₂:CO₂:H₂ (75:20:5, v/v/v) and the contents were filtered through a 4-layered cheesecloth.
3. Fresh rumen fluid was aliquoted to a 560 mL serum bottle, stoppered with rubber stopper, and brought outside the anaerobic chamber.
4. Concentrated fresh rumen fluid was filled with N₂:CO₂ (80:20, v/v) gas to 10 psig.
5. The ready probiotic mixtures were then added with 10 mL concentrated fresh rumen fluid using syringe.

### Step 5: Rumen-Probiotic Experiment

1. Serum bottles with rumen fluid, feed, and probiotic contents were incubated at 40° C., 75 rpm.
2. Each of the bottles were then treated in the following order:
   a. Bottle pressure was measured using a gas gauge in our lab gassing station system.
   b. 100 μL gas sample from the bottle headspace was taken for GC analysis.
   c. After 3 h, bottle pressure was measured and 100 μL gas sample from the bottle headspace was taken for GC analysis.
   d. The process (step 3) was then repeated every 3 h for total of 6 h, then every 6 h for total of 6 h, and then every 12 h for total of 36 h.

### Effects of Different Microbial Strains on Ruminal Methane Emission

Methane reduction effects of five animal-origin microbial strains, Table 1, were investigated in in-vitro rumen batch culture. In-vitro rumen batch culture is a surrogate system that simulates microbial fermentation process in rumen of ruminants.

TABLE 1
Probiotics/Additives List

| Probiotics/Additives | Final Concentration |
|---|---|
| Bacillus subtilis BE191105 | 1 × 10⁸ CFU per g feed |
| (ELA191105) | |
| Bacillus amyloliquefaciens | 1 × 10⁸ CFU per g feed |
| BE191006 (ELA191006) | |
| Bacillus amyloliquefaciens | 1 × 10⁸ CFU per g feed |
| BE202071 (ELA202071) | |
| Lactobacillus reuteri | 1 × 10⁸ CFU per g feed |
| BE3632 (strain 3632) | |
| Strain A | 1.43 × 10⁹ CFU per g feed |

The artificial saliva composition is shown in TABLE 2.

TABLE 2
Artificial Saliva Composition

| Compounds | Concentration | |
|---|---|---|
| NaCl | 8.04 | mmol/L |
| KCl | 8.18 | mmol/L |
| NaH₂PO₄•H₂O | 12.25 | mmol/L |
| Na₂HPO₄•7H₂O | 13.91 | mmol/L |
| NaHCO₃ | 95.24 | mmol/L |
| (NH₄)₂SO₄ | 7.14 | mmol/L |
| CaCl₂•2H₂O | 0.20 | mmol/L |
| MgCl₂•6H₂O | 0.34 | mmol/L |
| Na₃NTA | 10.00 | μmol/L |
| FeCl₃•6H₂O | 2.96 | μmol/L |
| CoCl₂•6H₂O | 0.42 | μmol/L |
| MnCl₂•4H₂O | 5.05 | μmol/L |

A summary of the cattle and feed used in the rumen experiment is as follows:

| | |
|---|---|
| Cattle breed | Holstein Friesian |
| Cattle type | Dairy |
| Rumen fluid sampling time | ~2 h after morning feeding |
| Feed composition | mixtures of concentrate, forage, |
| | silage, and hay grass |

The data summary is as follows:

| Bottle volume | ~270 | mL |
|---|---|---|
| Rumen fluid dilution factor | 1:2 (v/v) | |
| Diluted rumen fluid used | 30 | mL |
| Feed per bottle | 3 g (10% w/v) | |
| Probiotics, powder | 0.1 g (1 × 10⁸ CFU per g feed) | |
| Probiotics, liquid | 10-600 | μL |
| Control negative | 0.1 g CaCO₃ powder | |
| Control positive | 0.2 mM CHCl₃ | |
| Bottle headspace volume | ~240 | mL |

| | |
|---|---|
| Incubation | 40° C., 75 rpm |
| Measurement | bottle pressure and CH₄ via GC |
| | (100 μL injection volume) |
| Duration | 36 h (3 h sampling for 6 h, 6 h sampling |
| | for 6 h, and 12 h sampling for 36 h) |

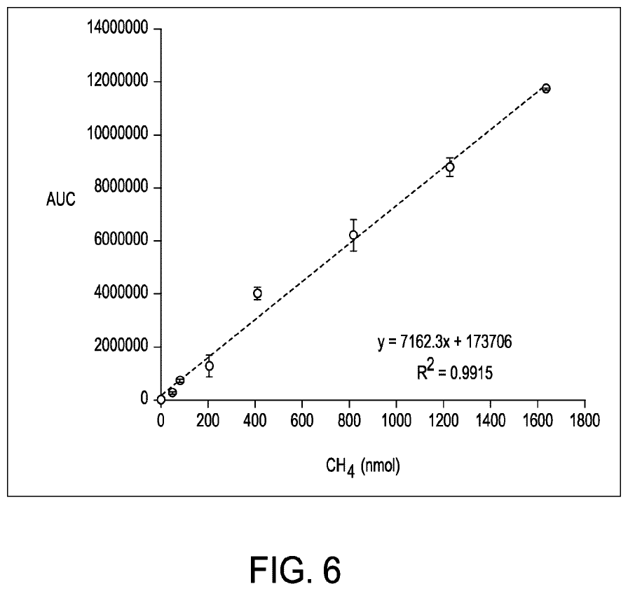

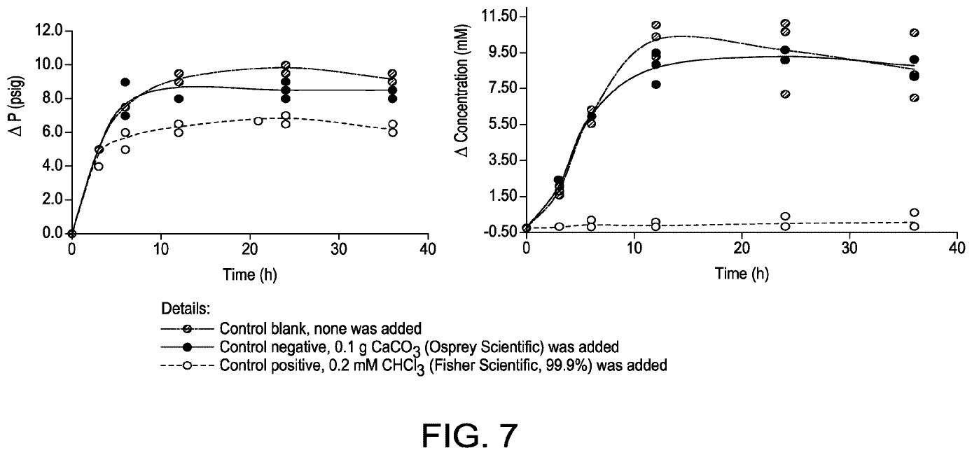

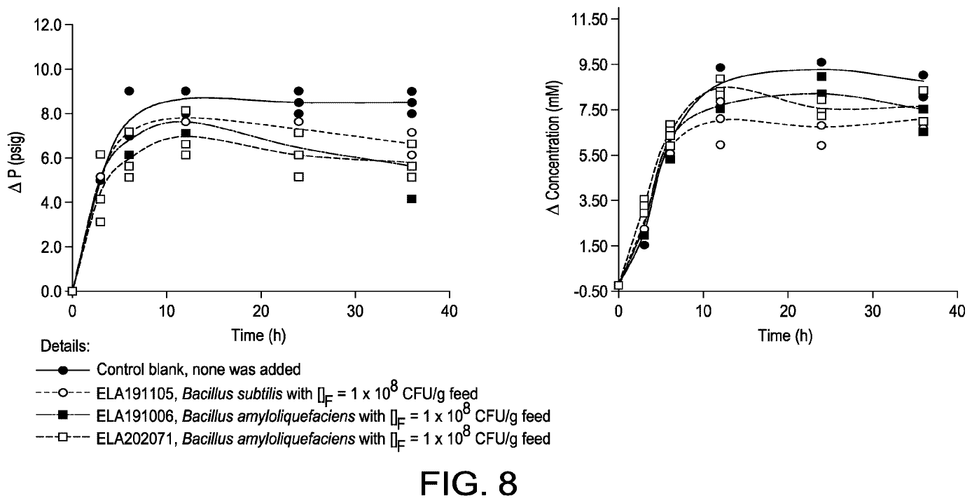

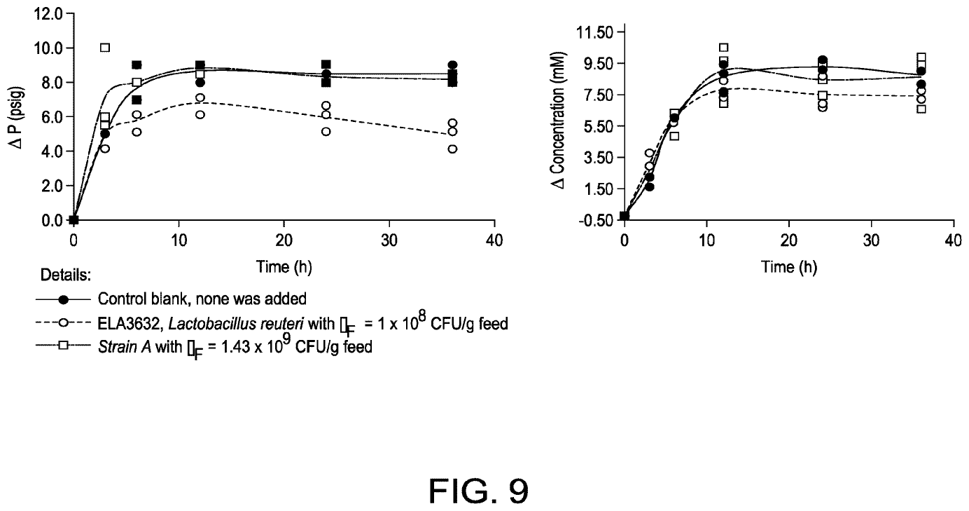

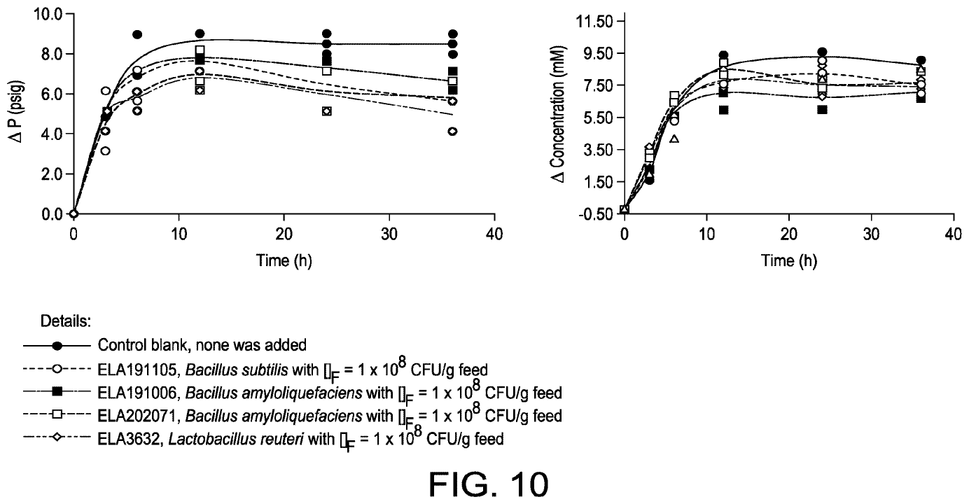

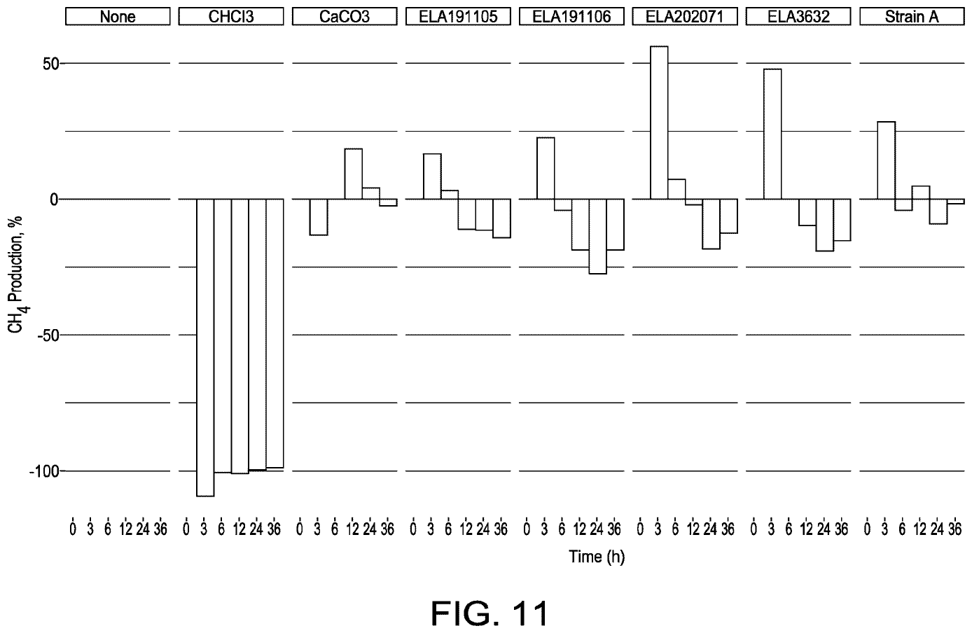

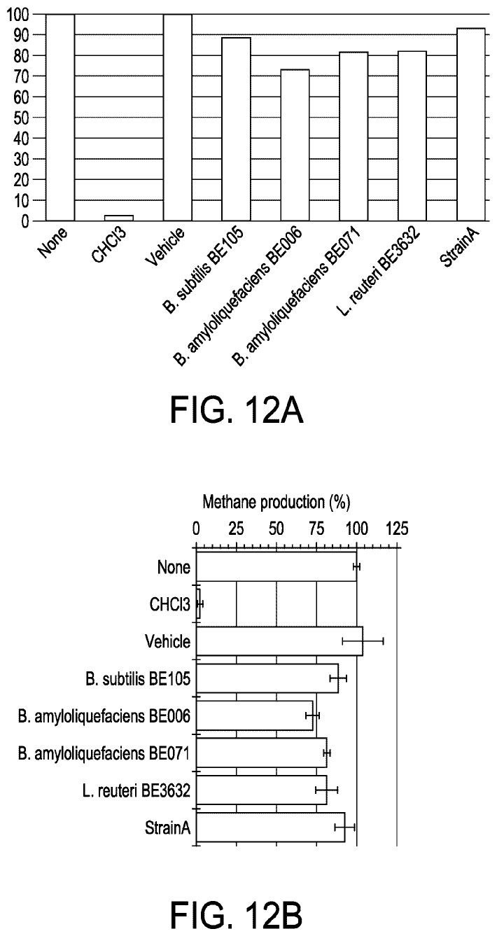

A methane standard curve representing the amount of methane gas injected into the gas chromatography (GC) system is provided in FIG. 6. Results with a control series are depicted in FIG. 7. Results with a Bacillus bacteria series are depicted in FIG. 8. Each of the Bacillus strains B. subtilis BE105 (BE191105/ELA191105)), B. amyloliquefaciens BE006 (BE191006/ELA191006)) and B. amyloliquefaciens BE071 (BE202071/ELA202071) are compared with control blank where none was added. Results with a Lactobacillus bacteria series are depicted in FIG. 9. Comparison results of Bacillus and Lactobacillus strains versus control are depicted in FIG. 10. Methane production (CH₄ production, %) is depicted in FIG. 11 for the various additives and bacteria strains tested. Methane production is defined as the increase (+ value) or decrease (− value) of methane production of a typical treatment when compared to a control blank (None). Methane Production from ex vivo rumen culture 24 h after supplementation of different probiotic strains is shown in FIGS. 12A and 12B.

All probiotics, except for other strain Strain A, reduced the methane production after 12 h of incubation and kept the methane production low in the next 30 h. Bottles with BE191006 (B. amyloliquefaciens) added had the lowest methane production compared to other probiotic treatments. Results suggested that the strains tested showed methane reduction effects when added into rumen cultures. Most methane reductions were observed after 6 hours of strains additions and peak at 24 hours. B. amyloliquefaciens BE191006 showed the highest level of methane reduction, ~25%, compared to the control, rumen culture without an addition of microbial strain.

Inhibition of methanogenic bacterial strains, particularly Methanobacterium bryantii, is evaluated in vitro. Growth of the methanogenic bacteria are evaluated. A reduction in bacterial growth is utilized in a kill type culture assay to determine the minimal inhibitory amount of one or more test strain, including one or more Bacillus or Lactobacillus strain, alone and in combinations, for methanogenic bacteria killing. In a first set of studies, Bacillus strains 06, 071, 105, alone and in combinations of two or three strains is tested. The Bacillus spp. strains are grown routinely in standard broth such as Lysogeny Broth (LB) and incubated at 37° C. overnight while shaking. Methanogenic bacteria, particularly Methanobacterium bryantii, is grown separately in an overnight culture to confluence. Bacillus are added to the methanogenic bacteria cultures and growth is assessed. In a separate set of studies Lactobacillus strains are evaluated alone and in combination. The cultures are observed for cell death. Assays are performed in duplicate.

### Example 2

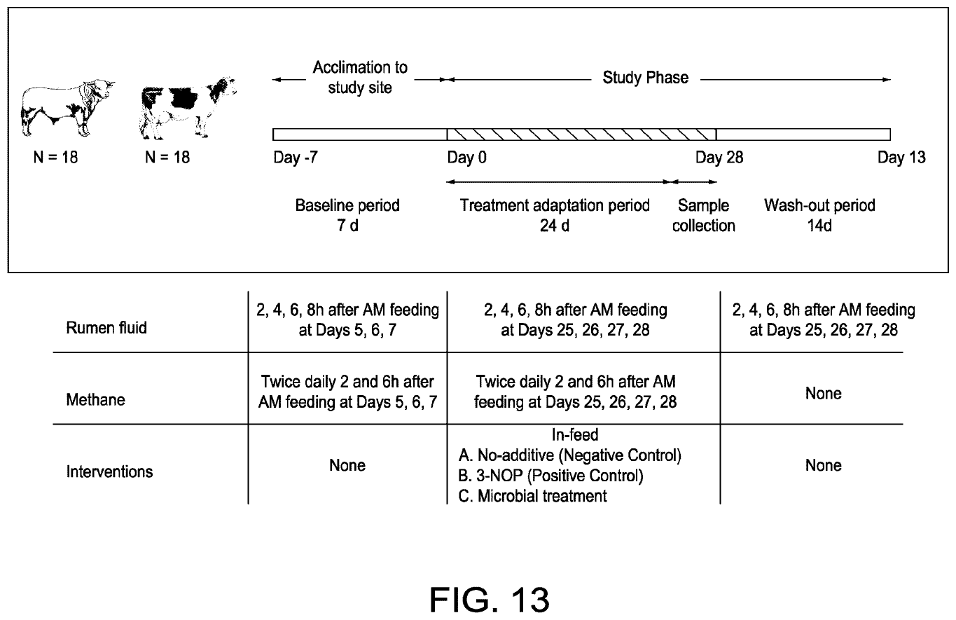

Beef cattle and dairy cattle are utilized in an in vivo in animal study to further evaluate the effect of administration of bacteria capable of reducing methane production in cattle performance. A randomized Latin square (3×3) crossover study design is depicted in FIG. 13. There is a baseline period of 7 days, followed by a Study Phase period which includes a 24 day Treatment Adaptation Period and a 4 day sample collection period to Day 28 of the study, followed by a 14 day Wash Out Period. Rumen fluid is evaluated throughout the study. Methane is assessed at Days 5, 6 and 7 of the baseline period and Days 25, 26, 27 and 28 of the Sample Collection period of the Study Phase. Interventions in feed include: A. No additive (negative control), B. 3-NOP (positive control) and C. Microbial treatment. With interventions in feed, rumen fluid is collected 2, 4, 6 and 8 hours after AM feeding, in this instance at Days 25, 26, 27 and 28 of the study.

Performance parameters for beef cattle are decreased Residual Feed Intake, Increased body weight, reduction of methane emission. Performance parameters for dairy cattle are increased milk volume, and protein and fat milk compositions.

### Example 3

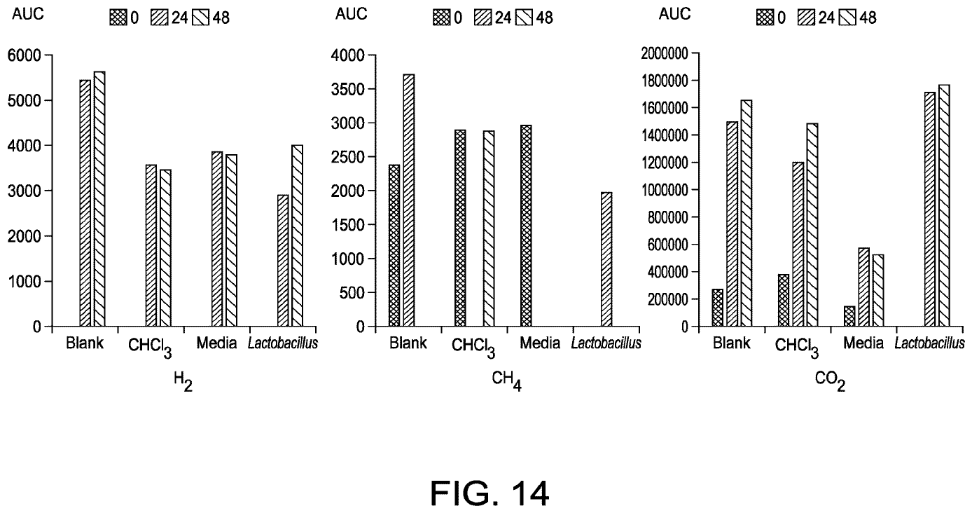

An in vitro rumen batch culture was utilized to evaluate the effect of bacteria (L. reuteri strain 3632 (BE3632)) on production of various gases in culture. Levels and amounts of H₂, CO₂ and CH₄ were assessed by gas chromatography. Results are shown in FIG. 14.

A significant reduction of methane CH₄ production is evident with L. reuteri BE3632 at 24 hours, with methane reduction by up to 50%. The methane reduction observed with L. reuteri BE3632 is greater than the reduction provided with the control addition of CHCl₃. The L. reuteri bacteria is shown to reduce H₂ production in the batch culture, particularly at 24 hours. CO₂ production is about the same in the presence of the L. reuteri bacteria versus the untreated sample.

### Example 4

Colonization by bacteria in rumen fluid was evaluated. This was conducted to demonstrate that the bacteria remain in and grow in the rumen environment. L. reuteri strain 3632 (BE3632) bacteria, which is not natively rumen-derived, was evaluated for colonization.

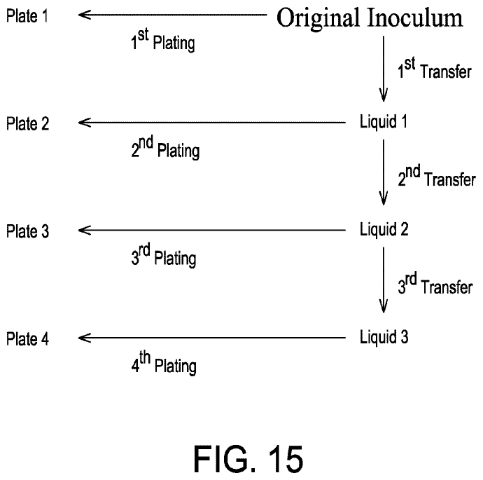

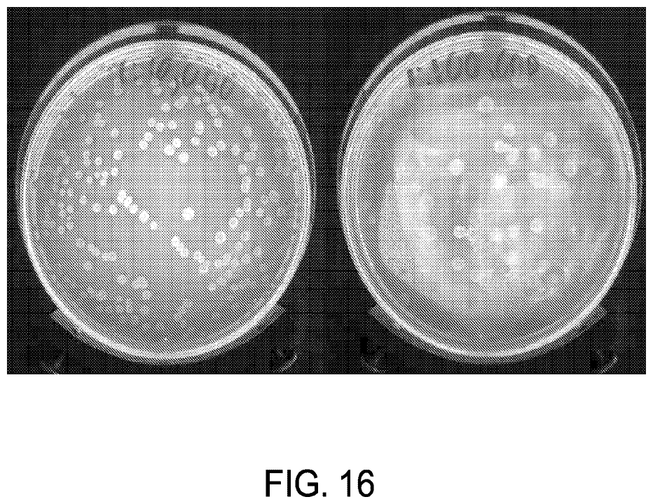

The process steps to evaluate L. reuteri BE3632 colonization in rumen fluid are depicted in FIG. 15. Starting with an original inoculum, which was sampled and plated to evaluate the number of bacteria, an inoculum was transferred in series up to 3 times. There was a period of two days between each transfer. At each transfer, a sample was taken for plating and the number of bacteria assessed. A sample of plate evaluations showing the L. reuteri BE3632 bacteria is provided in FIG. 16. Bacterial colonies of L. reuteri BE3632 are evident visually by their orange color. Colonies remained with and after transfer. After the second transfer, a 3-fold reduction of L. reuteri BE3632 bacteria counts was observed (data not shown). Further reduction was seen after the third transfer (data not shown), however some bacteria were retained. The L. reuteri BE3632 bacteria were retained and survived for numerous days (at least 6 days or nearly a week) with serial transfer in rumen fluid mimicking the rumen environment.

Media composition for the liquid cultures included rumen fluid, which was diluted with artificial saliva in a 1:1 ratio, plus dried cattle feed (cattle feed obtained from Virginia Tech Dairy Science Complex). Rumen fluid was collected from dairy cattle. Rumen fluid contains microbes (i.e. bacteria, fungi, protozoa—10¹¹ cells/mL), microbial metabolites (e.g. volatile fatty acids—mostly short chains, also contain medium and long-chain fatty acids, proteins, peptides), bovine saliva (we have saliva composition that we could share with you), micronutrients, and host proteins. pH-neutral (6.5-7). The rumen fluid composition is dependent on diet, age, breed (dairy vs cattle) and the rumen microbial population of the source animal. Rumen fluid components are described in, for example, Akula et al (Akula S et al (2023) Int J Mol Sci 24 (23): 16838). Cows produce saliva in very large quantities to lubricate and facilitate food processing. Bovine salivary proteins include carbonic anhydrase 6, a pH-stabilizing enzyme and the short palate, lung and nasal epithelium carcinoma-associated protein 2A (SPLUNC2A), also named bovine salivary protein 30 kDa (BSP30) or BPIFA2B, and secretory IgA. Liquid media was made by mixing 1.8 ml diluted BiomEdit rumen fluid with 0.2 g of dried cattle feed. Media composition for the plates was made up of Difco™ Lactobacilli MRS agar 1×, Bacto agar 2.5% (w/v) and tetracycline 50 μg/ml. L. reuteri BE3632 is resistant to tetracycline. Bottles, rubber stoppers, spatulas and beaker glasses used for making the liquid media were autoclaved before hand to minimize bacterial contamination. All plating and transfer were done anaerobically. The plate was incubated static anaerobically while the anaerobic liquid medium was incubated with shaking at 100 rpm. All incubation was performed at 40° C. for 48 hr. All dilution for plating was done using artificial saliva.

These results demonstrate that L. reuteri BE3632 is retained and survives in a rumen environment and is capable of colonization in the rumen of a ruminant animal.

### Example 5

The prior studies demonstrate that each of various specific isolated Bacillus strains and also Lactobacillus strains reduce methane production and alter methanogenesis, including in an in vitro system mimicking a native methanogenic (rumen) environment. In particular, each and all of Bacillus amyloliquefaciens strain BE191006, Bacillus amyloliquefaciens strain BE202071, Bacillus subtilis strain BE191105 and Lactobacillus reuteri strain 3632 reduce methane. Experiments were conducted to examine the compatibility of the Bacillus and Lactobacillus strains to further assess their use and application in combination to reduce methane emissions and alter methanogenesis.

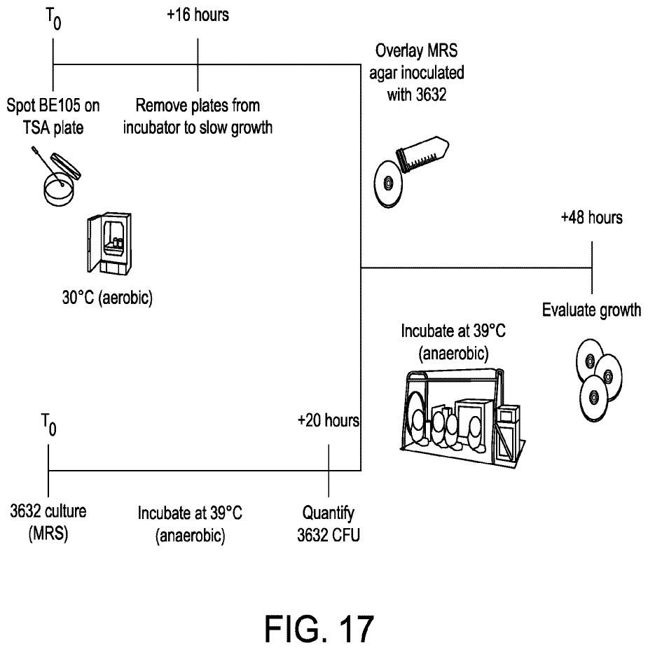

A depiction of an overlay experiment to examine strain compatibility is provided in FIG. 17. An evaluation of Bacillus subtilis strain BE191105 with Lactobacillus reuteri strain 3632 was conducted, as outlined in FIG. 17. The first strain (in this instance B. subtilis BE191105) is spotted on a plate (such as a TSA plate) and grown in aerobic conditions at 30° C. The second test strain (in this instance L. reuteri strain 3632) is grown separately in culture and incubated anaerobically at 39° C. for at least 20 hours. The second strain CFU is quantified. After at least 16 hours of growth, the second strain (in this instance L. reuteri strain 3632) is diluted to 10⁵ CFU and agar (such as MRS agar) is inoculated with the second strain and overlayed around the colony of the first strain ((in this instance B. subtilis BE191105). The plate is incubated anaerobically at 39° C. for at least 18, 24 or 48 hours and then the growth of the first and second strains is evaluated. In particular, inhibition of the first strains growth due to the presence of the second strain is assessed. In multiple technical replicates and also multiple biological replicates, no growth inhibition of the first strain (in this instance B. subtilis BE191105) by the second strain (in this instance L. reuteri strain 3632) was observed. The experiment is conducted similarly with other alternative strain combinations, including Bacillus amyloliquefaciens strain BE191006 or Bacillus amyloliquefaciens strain BE202071 as the first strain and Lactobacillus reuteri strain 3632 as the second strain.

This invention may be embodied in other forms or carried out in other ways without departing from the spirit or essential characteristics thereof. The present disclosure is therefore to be considered as in all aspects illustrated and not restrictive, the scope of the invention being indicated by the appended Claims, and all changes which come within the meaning and range of equivalency are intended to be embraced therein.

Various references are cited throughout this Specification, each of which is incorporated herein by reference in its entirety.

1. A probiotic or microbiome modulator composition comprising at least one bacteria strain selected from a Bacillus species and Lactobacillus species, or combinations thereof, wherein the composition reduces methane gas emissions from an animal when an effective amount is administered to the animal, as compared to an animal not administered the composition, wherein the composition comprises one or more bacterial strain selected from a Bacillus amyloliquefaciens strain, a Bacillus subtilis strain, and a Lactobacillus strain, wherein the Lactobacillus strain is a Lactobacillus reuteri strain.

2. (canceled)

3. (canceled)

4. The composition of claim 1 comprising at least one of Lactobacillus reuteri strain 3632 or a Lactobacillus strain having at least 90% identity, 95% identity, 97% identity, 98% identity, 99% identity in genomic sequence to the sequence of at least one of SEQ ID NOs: 51-57; Bacillus amyloliquefaciens strain BE191006 or Bacillus amyloliquefaciens strain BE191024 or a Bacillus strain having at least 90% identity, 95% identity, 97% identity, 98% identity, 99% identity in genomic sequence to the sequence of SEQ ID NO: 27; Bacillus amyloliquefaciens strain BE202071 or a Bacillus strain having at least 90% identity, 95% identity, 97% identity, 98% identity, 99% identity in genomic sequence to the sequence of SEQ ID NO: 42; and Bacillus subtilis strain BE191105 or a Bacillus strain having at least 90% identity, 95% identity, 97% identity, 98% identity, 99% identity in genomic sequence to one or more of SEQ ID NO: 1, 2, 3, 4 and 5.

5. The composition of claim 1 comprising at least one of Lactobacillus reuteri strain 3632 corresponding to ATCC Patent Deposit Number PTA-126788, Bacillus amyloliquefaciens strain BE191006 (also denoted as ELA191006) corresponding to ATCC deposit PTA-127065, Bacillus amyloliquefaciens strain BE191024 corresponding to ATCC Patent Deposit Number PTA-126784, Bacillus amyloliquefaciens strain BE202071 (also denoted as ELA202071) corresponding to ATCC deposit PTA-127064, and Bacillus subtilis strain BE191105 (also denoted as ELA191105) corresponding to ATCC deposit PTA-126786.

6. The composition of claim 1 comprising at least two of Lactobacillus reuteri strain 3632 or a Lactobacillus strain having at least 90% identity, 95% identity, 97% identity, 98% identity, 99% identity in genomic sequence to the sequence of at least one of SEQ ID NOs: 51-57; Bacillus amyloliquefaciens strain BE191006 or Bacillus amyloliquefaciens strain BE191024 or a Bacillus strain having at least 90% identity, 95% identity, 97% identity, 98% identity, 99% identity in genomic sequence to the sequence of SEQ ID NO: 27; Bacillus amyloliquefaciens strain BE202071 or a Bacillus strain having at least 90% identity, 95% identity, 97% identity, 98% identity, 99% identity in genomic sequence to the sequence of SEQ ID NO: 42; and Bacillus subtilis strain BE191105 or a Bacillus strain having at least 90% identity, 95% identity, 97% identity, 98% identity, 99% identity in genomic sequence to one or more of SEQ ID NO: 1, 2, 3, 4 and 5.

7. (canceled)

8. The composition of claim 1, further comprising one or more anti-methanogenic compound, wherein the anti-methanogenic compound is selected from an inhibitor of a rate-limiting enzyme in methanogenesis pathway, an inhibitor of methanogens lipid biosynthesis, or a microbiome modulator.

9. A method for reducing methane gas production or methane gas emissions from an animal comprising administering an effective amount of a composition of claim 1 including at least one probiotic or microbiome modulator selected from a Bacillus species, Lactobacillus species, or combinations thereof, to an animal.

10. The method of claim 9 wherein the animal is a ruminant animal, and wherein the ruminant animal is selected from bovines such as cattle, bison, American buffalo and water buffalos, goats, sheep, giraffes, deer, gazelles, and antelopes.

11. (canceled)

12. The method of claim 9 wherein the animal is a non-ruminant animal, and wherein the non-ruminant animal is selected from poultry, pigs, birds, aquatic animals such as fish and crustaceans, horses, dogs, cats, and humans.

13. (canceled)

14. The method of claim 9 comprising one or more bacterial strain selected from a Bacillus amyloliquefaciens strain, a Bacillus subtilis strain, and a Lactobacillus strain, wherein the Lactobacillus strain is a Lactobacillus reuteri strain.

15. The method of claim 9, wherein the composition or a fermentation product of the composition is administered, and wherein composition or a fermentation product of the composition is formulated as animal feed.

16. (canceled)

17. The method of claim 9, wherein the composition comprises at least one of Lactobacillus reuteri strain 3632 or a Lactobacillus strain having at least 90% identity, 95% identity, 97% identity, 98% identity, 99% identity in genomic sequence to the sequence of at least one of SEQ ID NOs: 51-57; Bacillus amyloliquefaciens strain BE191006 or Bacillus amyloliquefaciens strain BE191024 or a Bacillus strain having at least 90% identity, 95% identity, 97% identity, 98% identity, 99% identity in genomic sequence to the sequence of SEQ ID NO: 27; Bacillus amyloliquefaciens strain BE202071 or a Bacillus strain having at least 90% identity, 95% identity, 97% identity, 98% identity, 99% identity in genomic sequence to the sequence of SEQ ID NO: 42; and Bacillus subtilis strain BE191105 or a Bacillus strain having at least 90% identity, 95% identity, 97% identity, 98% identity, 99% identity in genomic sequence to one or more of SEQ ID NO: 1, 2, 3, 4 and 5.

18. The method of claim 9, comprising at least one of Bacillus amyloliquefaciens strain BE191006 (also denoted as ELA191006) corresponding to ATCC deposit PTA-127065 or Bacillus amyloliquefaciens strain BE191024 corresponding to ATCC Patent Deposit Number PTA-126784, Bacillus amyloliquefaciens strain BE202071 (also denoted as ELA202071) corresponding to ATCC deposit PTA-127064, Bacillus subtilis strain BE191105 (also denoted as ELA191105) corresponding to ATCC deposit PTA-126786, and Lactobacillus reuteri strain 3632 corresponding to ATCC Patent Deposit Number PTA-126788.

19. The method of claim 9, wherein the composition comprises at least two isolated strains selected from Bacillus amyloliquefaciens strain BE191024, Bacillus amyloliquefaciens strain BE191006, Bacillus subtilis strain BE191105, and Lactobacillus reuteri strain 3632.

20. The method of claim 9, further comprising administering one or more anti-methanogenic compound, wherein the anti-methanogenic compound is selected from an inhibitor of a rate-limiting enzyme in methanogenesis pathway, an inhibitor of methanogens lipid biosynthesis, or a microbiome modulator.

21. (canceled)

22. The method of claim 9, wherein the animal is a human, and wherein the human has an intestinal disorder, obesity, or a high body mass index (BMI).

23. (canceled)

24. A method for reducing methane emission or production by methanogenic organisms selected from methanogenic Archaea or methanogenic bacteria in the environment, in a culture or in a process comprising administering the composition of claim 1 or a fermentation product of the composition.

25. The method of claim 24, wherein the environment, culture or process is selected from manure, litter, ponds, an aerobic digester, and waste treatment.

26. The method of claim 24 for reducing methane emission or production in manure by combining or adding to the manure the composition of claim 1 or a fermentation product of the composition.

27. A postbiotic comprising a fermentation product of the composition of claim 1, wherein the postbiotic reduces methane production, alters methanogenesis, and/or reduces greenhouse emissions, wherein the postbiotic is formulated as a feed additive, a food or food additive, or otherwise for an animal, and wherein the animal is selected from bovines such as cattle, bison, American buffalo and water buffalos, goats, sheep, giraffes, deer, gazelles, antelopes, poultry, pigs, birds, aquatic animals such as fish and crustaceans, horses, dogs, cats and humans.

28. (canceled)

29. (canceled)

30. (canceled)

31. (canceled)
